(19) United States
(12) Patent Application Publication
(10) Pub. No.: US 2021/0121372 A1
(43) Pub. Date: Apr. 29, 2021

(54) SPRAYABLE COSMETIC AGENT I

(71) Applicant: Henkel AG & Co. KGaA, Duesseldorf (DE)

(72) Inventors: DIANE METTEN, Hamburg (DE); RENE SCHEFFLER, Ellerau (DE); JULIA BIBIANE LANGE, Bad Bramstedt (DE); CYRIELLE MARTINEZ, Hamburg (DE); THORSTEN KNAPPE, Schenefeld (DE); ROLF BAYERSDOERFER, Hamburg (DE)

(73) Assignee: Henkel AG & Co. KGaA, Duesseldorf (DE)

(21) Appl. No.: 16/497,928

(22) PCT Filed: Mar. 9, 2018
(86) PCT No.: PCT/EP2018/055893

§ 371 Date: Sep. 26, 2019

(30) Foreign Application Priority Data
Mar. 31, 2017 (DE) 10 2017 205 558.8
Nov. 21, 2017 (DE) 10 2017 220 773.6

(51) Int. Cl. A61K 8/02; A61K 8/04; A61K 8/34; A61K 8/73; A61K 8/81; A61Q 5/06

(52) U.S. Cl. CPC A61K 8/027; A61K 8/046; A61K 8/34; A61K 8/731; A61K 8/732; A61K 8/8158; A61K 2800/87; A61K 8/8182; A61K 8/8135; A61K 8/8147; A61Q 5/06; A61K 2800/412; A61K 2800/43; A61K 8/8176

(57) ABSTRACT

A cosmetic composition for the cosmetic treatment of keratin fibers is provided. The composition includes:
(a) fibers,
(b) ethanol,
(c) at least one polymer, and
(d) propellant.

### CROSS-REFERENCE TO RELATED APPLICATION

This application is a U.S. National-Stage entry under 35 U.S.C. § 371 based on International Application No. PCT/EP2018/055893, filed Mar. 9, 2018, which was published under PCT Article 21(2), which claims priority to German Application No. 10 2017 220 773.6, filed Nov. 21, 2017 and which claims priority to German Application No. 10 2017 205 558.8, filed Mar. 31, 2017, which are all hereby incorporated in their entirety by reference.

### TECHNICAL FIELD

The present application relates to sprayable cosmetic compositions for the treatment of keratin fibers, which compositions, in addition to a solvent and a propellant, also contain fibers. These compositions are suitable for a wide range of cosmetic uses, but in particular for the temporary shaping of keratin fibers. The application also relates to the cosmetic use of these compositions and to hair cosmetic methods employing these compositions.

### BACKGROUND

Styling products for shaping keratinous fibers have long been known are used in various embodiments in order to structure, refresh and fix hairstyles, which in the case of many hair types can be achieved only with the use of firming active substances. Here, both hair treatment products which are used to permanently shape and also those used to temporarily shape the hair play an important role. Temporary shaping with a good hold without adversely affecting the healthy appearance of the hair, such as its shine, can be achieved for example by hairsprays, hair waxes, hair gels, blow-drying, etc.

Corresponding compositions for temporarily shaping usually contain synthetic polymers as shaping component. Preparations that contain a polymer can be applied to the hair by employing propellant gases or by a pump mechanism. Hair gels and hair waxes, by contrast, generally are not applied directly to the hair, but instead are distributed over the hair by employing a comb or by the hands.

Known forms of temporary styling products often cannot be dosed with satisfactory accuracy. For example, hair gels, hair creams and hair waxes thus can still only be distributed with difficulty once they have been applied to the hair.

Hairsprays can be distributed uniformly over the hair, however there is also considerable scope for improvement with regard to the application properties of these sprayable hair cosmetics.

### BRIEF SUMMARY

Cosmetic compositions for cosmetic treatment of keratin fibers are provided. In an exemplary embodiment, a cosmetic composition for cosmetic treatment of keratin fibers includes fibers, ethanol, at least one polymer, and a propellant.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

The object of the present disclosure was to provide sprayable hair cosmetics which are exemplified, in comparison to conventional preparations, by improved application properties, in particular in improved spray pattern. It has been found that this object can be achieved by the addition of fibers to the sprayable cosmetic preparations.

The following is provided by the present disclosure:

A cosmetic composition for the cosmetic treatment of keratin fibers, said composition comprising:
(a) fibers,
(b) ethanol,
(c) at least one polymer, and
(d) propellant.

The cosmetic composition as contemplated herein, wherein the cosmetic composition also contains
(e) from about 0 to about 10% by weight of dye(s)
(f) from about 0 to about 10% by weight of pigment(s), with the provision that
   the sum of the amounts of ingredients (e) and (0 is from about 0.0001 to about 10% by weight, in relation to the total weight of the cosmetic composition.

The cosmetic composition as contemplated herein, wherein the weight share of the fibers in the total weight of the cosmetic composition is from about 0.1 to about 5.0, preferably from about 0.1 to about 2.0% by weight, particularly preferably from about 0.1 to about 1.0% by weight, and in particular from about 0.1 to about 0.5% by weight.

The cosmetic composition as contemplated herein, wherein the fibers are selected from the group of fibers produced from natural fiber materials.

The cosmetic composition as contemplated herein, wherein the fibers are selected from the group of plant fibers, in particular cellulose fibers, preferably cotton fibers, flax fibers and kapok fibers.

The cosmetic composition as contemplated herein, wherein the fibers are selected from the group of animal fibers, preferably silk fibers, cashmere fibers and wool fibers.

The cosmetic composition as contemplated herein, wherein the fibers are selected from the group of fibers produced from synthetic fiber materials.

The cosmetic composition as contemplated herein, wherein the fibers are selected from the group of polyamide fibers, polyester fibers, polylactic acid fibers and polyhydroxyalkanoate fibers.

The cosmetic composition as contemplated herein, wherein the fibers have a length of from about 1.0 to about 200 μm, preferably from about 2.0 to about 150 μm, and in particular from about 5.0 to about 100 μm.

The cosmetic composition as contemplated herein, wherein the weight share of the ethanol in the total weight of the cosmetic composition is from about 20 to about 50% by weight, preferably from about 20 to about 45% by weight, and in particular from about 25 to about 40% by weight.

The cosmetic composition as contemplated herein, wherein the weight share of the at least one polymer in the total weight of the cosmetic composition is from about 0.5 to about 8.0% by weight, preferably from about 1.0 to about 6.0% by weight, and in particular from about 2.0 to about 4.0% by weight.

The cosmetic composition as contemplated herein, wherein the polymer is selected from the group of polyvinylpyrrolidone and vinylpyrrolidone/vinyl acetate copolymer.

The cosmetic composition as contemplated herein, wherein the polymer is selected from the group of copolymers of i) N-tert-octylacrylamide, ii) acrylic acid, iii) tert.-butylaminoethyl methacrylate and optionally further monomers.

The cosmetic composition as contemplated herein, wherein the weight share of the propellant in the total weight of the cosmetic composition is from about 30 to about 70% by weight, preferably from about 35 to about 65% by weight, and in particular from about 40 to about 60% by weight.

The cosmetic composition as contemplated herein, wherein the composition comprises a propellant from the group propane, propane/butane mixtures, and dimethyl ether, in particular dimethyl ether.

The cosmetic composition as contemplated herein, wherein the composition comprises at least one ester oil as further constituent.

The cosmetic composition as contemplated herein, wherein the composition, in relation to its total weight, comprises from about 0.01 to about 1.0% by weight, preferably from about 0.02 to about 0.6% by weight, and in particular from about 0.03 to about 0.2% by weight of ester oil.

The cosmetic composition as contemplated herein, wherein the fibers are hydrophobized.

The cosmetic composition as contemplated herein, wherein the fibers have an oil absorption capacity (about 20° C.) of from about 0.2 to about 0.8 g jojoba oil/g, preferably from about 0.3 to about 0.7 g jojoba oil/g, particularly preferably from about 0.4 to about 0.6 g jojoba oil/g.

The cosmetic composition as contemplated herein, wherein the fibers are hydrophilized.

The cosmetic composition as contemplated herein, wherein the fibers, in relation to their net weight, have a water adsorption capacity (about 20° C.) above about 5.0% by weight, preferably above about 7.0% by weight, and in particular above about 9.0% by weight.

The cosmetic composition as contemplated herein, wherein it contains—in relation to its total weight—from about 0.0001 to about 10% by weight, preferably from about 0.01 to about 9.5% by weight, more preferably from about 0.05 to about 9% by weight, even more preferably from about 0.1 to about 8.5% by weight, and in particular from about 0.25 to about 8% by weight of dye(s).

The cosmetic composition as contemplated herein, wherein it contains—in relation to its total weight—from about 0.0001 to about 10% by weight, preferably from about 0.01 to about 9.5% by weight, more preferably from about 0.05 to about 9% by weight, even more preferably from about 0.1 to about 8.5% by weight, and in particular from about 0.25 to about 8% by weight of pigment(s).

The cosmetic composition as contemplated herein, wherein it contains at least one pigment from the group CI12490, CI14700, CI14720, CI15510, CI15985, CI45380, CI47005, CI60730, CI61565, CI73360, CI74160, CI77007, CI77019, CI77288, CI77289, CI77491, CI77492, wherein the total amount of pigment(s) from this group is from about 0.0001 to about 10% by weight, preferably from about 0.01 to about 9.5% by weight, more preferably from about 0.05 to about 9% by weight, even more preferably from about 0.1 to about 8.5% by weight, and in particular 0.25 to 8% by weight, in each case in relation to the total weight of the cosmetic composition.

The cosmetic composition as contemplated herein, wherein it contains, as further constituent, from about 1.0 to about 10% by weight, preferably from about 2.0 to about 6.0% by weight, and in particular from about 3.0 to about 5.0% by weight of a starch compound, in each case in relation to the total weight of the cosmetic composition.

The cosmetic composition as contemplated herein, wherein it contains ingredients (e) and/or (0 in the form of a starch-dye compound and/or starch-pigment compound, preferably in the form of dyed and/or pigmented rice starch.

The cosmetic composition as contemplated herein, wherein it contains the ingredients (a) and (e) and/or (a) and (f) in the form of a fiber-dye compound and/or a fiber-pigment compound, preferably in the form of dyed and/or pigmented fibers.

A cosmetic product comprising
a) a cosmetic composition as contemplated herein
b) an aerosol-dispensing container.

The product as contemplated herein, wherein the product has a spray rate above about 4.0 g/10 s, preferably above about 6.0 g/10 s, and in particular from about 6.5 to about 10.0 g/10 s.

Use of a cosmetic composition as contemplated herein for the temporary shaping of keratin fibers.

A method for the temporary shaping of keratin fibers, in particular human hair, in which the cosmetic composition as contemplated herein is applied to keratin fibers.

The cosmetic composition contains fibers as its first essential constituent. The addition of these fibers influences the application properties of the sprayable hair cosmetic in an unanticipated manner. The spray pattern and the application of the sprayed cosmetic are more uniform. In a standardized test assembly the addition of the fibers at the same pressure in the spraying device and at the same distance from the surface that is to be sprayed additionally results in a surprising enlargement of the spray area that is reached.

Oriented, flexible structures formed from fiber material with a ration of length to diameter above about 3:1, preferably above about 5:1, and in particular above about 10:1 are suitable as fibers. Fibers cannot take up compressive forces in the longitudinal direction, but instead only tensile forces.

The length of fibers that are used with preference ranges from about 1.0 to about 200 μm, preferably from about 2.0 to about 150 μm, and in particular from about 5.0 to about 100 μm. Of course, however, fibers of greater fiber length, for example fibers with a length above about 200 μm or above about 300 μm, can also be used.

The fiber can be of natural or synthetic origin. All fibers that are obtained from plant, animal or mineral material without any chemical modification are considered to be natural fibers.

Plant fibers occur in plants as a vascular bundle in the stalk, in the stem, in the bark or in the form of seed extensions. Plant fibers generally consist predominantly of cellulose. Corresponding fibers are amalgamated under the name cellulose fibers.

In accordance with common subdivisioning, a distinction is made in the case of plant fibers between seed fibers, bast fibers, leaf fibers and fruit fibers. The group of seed fibers for example includes cotton fibers, kapok, crown flower seed fibers, or populus fruit seed fibers. The group of bast fibers includes, amongst other things, bamboo fibers, stinging nettles, hemp fibers, jute, and flax fibers. Sisal is considered to belong to the leaf fibers, whereas coconut fibers belong to the group of fruit fibers.

In the case of animal fibers a distinction can be made, amongst other things, between fibers from silk glands and fibers originating from hair follicles. Fibers from silk glands include silk, for example. The group of fibers originating from hair follicles includes, amongst other things, wool, alpaca, camel hair, angora, cashmere, mohair, yak hair, goat hair, cattle hair or horsehair.

In a preferred embodiment the fibers used in the cosmetic compositions are selected from the group of fibers formed from natural fiber materials. The use of cellulose fibers, in particular cotton fibers, flax fibers and kapok fibers, is particularly preferred. The use of animal fibers from the group of silk fibers, cashmere fibers and wool fibers is also preferred. The use of cellulose fibers is particularly preferred.

Alternatively or in combination with the above-described natural fibers, fibers based on synthetic fiber materials can also be used in the cosmetic compositions.

The use of synthetic fiber materials obtained by modification of plant or animal starting materials is particularly preferred. The group of these preferred fibers includes, for example, fibers formed from viscose, polylactic acid, alginate, chitin or chitosan.

Further preferred synthetic fiber materials are polyamides, polyesters and polyhydroxyalkanoates.

Proportions by weight of the fibers in the total weight of the cosmetic composition from about 0.1 to about 5.0, preferably from about 0.1 to about 2.0% by weight, particularly preferably from about 0.1 to about 1.0% by weight, and in particular from about 0.1 to about 0.5% by weight have proven to be particularly advantageous for the technical and cosmetic effect.

As their second essential constituent, the cosmetic compositions contain ethanol. The weight share of the ethanol in the total weight of the cosmetic composition is preferentially from about 45 to about 70% by weight, preferably from about 40 to about 65% by weight, and in particular from about 50 to about 60% by weight. The fibers are suspended in the ethanol.

The third essential constituent of the cosmetic composition is the at least one polymer. For the applicability and cosmetic effect of the compositions, it has proven to be advantageous to limit the weight share of the polymer in the total weight of the composition to from about 0.5 to about 8.0% by weight, preferably from about 1.0 to about 6.0% by weight, and in particular from about 2.0 to about 4.0% by weight.

Polymers are used in the cosmetic compositions for example on account of their firming and/or film-forming properties. The use of film-forming polymers is particularly preferred on account of their cosmetic effect.

Permanently and also temporarily cationic, non-ionic or amphoteric polymers are suitable as polymers. The film-forming polymers can be of synthetic or natural origin.

Examples of conventional polymers are Acrylamide/Ammonium Acrylate Copolymer, Acrylamides/DMAPA Acrylates/Methoxy PEG Methacrylate Copolymer, Acrylamidopropyltrimonium Chloride/Acrylamide Copolymer, Acrylamidopropyltrimonium Chloride/Acrylates Copolymer, Acrylates/Acetoacetoxyethyl Methacrylate Copolymer, Acrylates/Acrylamide Copolymer, Acrylates/Ammonium Methacrylate Copolymer, Acrylates/t-Butylacrylamide Copolymer, Acrylates Copolymer, Acrylates/C1-2 Succinates/Hydroxyacrylates Copolymer, Acrylates/Lauryl Acrylate/Stearyl Acrylate/Ethylamine Oxide Methacrylate Copolymer, Acrylates/Octylacrylamide Copolymer, Acrylates/Octylacrylamide/Diphenyl Amodimethicone Copolymer, Acrylates/Stearyl Acrylate/Ethylamine Oxide Methacrylate Copolymer, Acrylates/VA Copolymer, Acrylates/VP Copolymer, Adipic Acid/Diethylenetriamine Copolymer, Adipic Acid/Dimethylaminohydroxypropyl Diethylenetriamine Copolymer, Adipic Acid/Epoxypropyl Diethylenetriamine Copolymer, Adipic Acid/Isophthalic Acid/Neopentyl Glycol/Trimethylolpropane Copolymer, Allyl Stearate/VA Copolymer, Aminoethylacrylate Phosphate/Acrylates Copolymer, Aminoethylpropanediol-Acrylates/Acrylamide Copolymer, Aminoethylpropanediol-AMPD-Acrylates/Diacetoneacrylamide Copolymer, Ammonium VA/Acrylates Copolymer, AMPD-Acrylates/Diacetoneacrylamide Copolymer, AMP-Acrylates/Allyl Methacrylate Copolymer, AMP-Acrylates/C1-18 Alkyl Acrylates/C1-8 Alkyl Acrylamide Copolymer, AMP-Acrylates/Diacetoneacrylamide Copolymer, AMP-Acrylates/Dimethylaminoethylmethacrylate Copolymer, Bacillus/Rice Bran Extract/Soybean Extract Ferment Filtrate, Bis-Butyloxyamodimethicone/PEG-60 Copolymer, Butyl Acrylate/Ethylhexyl Methacrylate Copolymer, Butyl Acrylate/Hydroxypropyl Dimethicone Acrylate Copolymer, Butylated PVP, Butyl Ester of Ethylene/MA Copolymer, Butyl Ester of PVM/MA Copolymer, Calcium/Sodium PVM/MA Copolymer, Corn Starch/Acrylamide/Sodium Acrylate Copolymer, Diethylene Glycolamine/Epichlorohydrin/Piperazine Copolymer, Dimethicone Crosspolymer, Diphenyl Amodimethicone, Ethyl Ester of PVM/MA Copolymer, Hydrolyzed Wheat Protein/PVP Crosspolymer, Isobutylene/Ethylmaleimide/Hydroxyethylmaleimide Copolymer, Isobutylene/MA Copolymer, Isobutylmethacrylate/Bis-Hydroxypropyl Dimethicone Acrylate Copolymer, Isopropyl Ester of PVM/MA Copolymer, Lauryl Acrylate Crosspolymer, Lauryl Methacrylate/Glycol Dimethacrylate Crosspolymer, MEA-Sulfite, Methacrylic Acid/Sodium Acrylamidomethyl Propane Sulfonate Copolymer, Methacryloyl Ethyl Betaine/Acrylates Copolymer, Octylacrylamide/Acrylates/Butylaminoethyl Methacrylate Copolymer, PEG/PPG-25/25 Dimethicone/Acrylates Copolymer, PEG-8/SMDI Copolymer, Polyacrylamide, Polyacrylate-6, Polybeta-Alanine/Glutaric Acid Crosspolymer, Polybutylene Terephthalate, Polyester-1, Polyethylacrylate, Polyethylene Terephthalate, Polymethacryloyl Ethyl Betaine, Polypentaerythrityl Terephthalate, Polyperfluoroperhydrophenanthrene, Polyquatemium-1, Polyquatemium-2, Polyquatemium-4, Polyquatemium-5, Polyquarternium-6, Polyquatemium-7, Polyquatemium-8, Polyquatemium-9, Polyquatemium-10, Polyquatemium-11, Polyquatemium-12, Polyquatemium-13, Polyquatemium-14, Polyquatemium-15, Polyquatemium-16, Polyquatemium-17, Polyquatemium-18, Polyquatemium-19, Polyquatemium-20, Polyquatemium-22, Polyquatemium-24, Polyquatemium-27, Polyquatemium-28, Polyquatemium-29, Polyquatemium-30, Polyquatemium-31, Polyquatemium-32, Polyquatemium-33, Polyquatemium-34, Polyquatemium-35, Polyquatemium-36, Polyquatemium-39, Polyquatemium-45, Polyquatemium-46, Polyquatemium-47, Polyquatemium-48, Polyquatemium-49, Polyquatemium-50, Polyquatemium-55, Polyquatemium-56, Polysilicone-9, Polyurethane-1, Polyurethane-6, Polyurethane-10, Polyvinyl Acetate, Polyvinyl Butyral, Polyvinylcaprolactam, Polyvinylformamide, Polyvinyl Imidazolinium Acetate, Polyvinyl Methyl Ether, Potassium Butyl Ester of PVM/MA Copolymer, Potassium Ethyl Ester of PVM/MA Copolymer, PPG-70 Polyglyceryl-10 Ether, PPG-12/SMDI Copolymer, PPG-51/SMDI Copolymer, PPG-10 Sorbitol, PVM/MA Copolymer, PVP, PVPNA/Itaconic Acid Copolymer, PVP/VA/Vinyl Propionate Copolymer, Rhizobian Gum, Rosin Acrylate, Shellac, Sodium Butyl Ester of PVM/MA Copolymer, Sodium Ethyl Ester of PVM/MA Copolymer, Sodium Polyacrylate, Sterculia Urens Gum, Terephthalic Acid/Isophthalic Acid/Sodium Isophthalic Acid Sulfonate/Glycol Copolymer, Trimethylolpropane Triacrylate, Trimethylsiloxysilylcarbamoyl Pullulan, VA/Crotonates Copolymer, VA/Crotonates/Methacryloxybenzophenone-1 Copolymer, VA/Crotonates/Vinyl Neodecanoate Copolymer, VA/Crotonates/Vinyl Propionate Copolymer, VA/DBM Copolymer, VA/Vinyl Butyl Benzoate/Crotonates Copolymer, Vinylamine/Vinyl Alcohol Copolymer, Vinyl Caprolactam/VP/Dimethylaminoethyl Methacrylate Copolymer, VP/Acrylates/Lauryl Methacrylate Copolymer, VP/Dimethylaminoethylmethacrylate Copolymer, VP/DMAPA Acrylates Copolymer, VP/Hexadecene Copolymer, VP/VA Copolymer, VP/Vinyl Caprolactam/DMAPA Acrylates Copolymer, Yeast Palmitate and Styrene/VP Copolymer.

The polymer is particularly preferably selected from the group of non-ionic polymers. Suitable non-ionic polymers are, for example:
Polyvinylpyrrolidones, as sold for example under the name Luviskol® (BASF).
Vinylpyrrolidone/vinyl ester copolymers, as sold for example under the name Luviskol® (BASF). Luviskol® VA 64 and Luviskol® VA 73, each vinylpyrrolidone/vinyl acetate copolymers, are preferred non-ionic polymers.
Cellulose ethers, such as hydroxypropyl cellulose, hydroxyethyl cellulose and methylhydroxypropyl cellulose, as sold for example under the trade names Culminal® and Benecel® (AQUALON).
Shellac.
Siloxanes. These siloxanes can be both water-soluble and water-insoluble. Both volatile and non-volatile siloxanes are suitable, wherein compounds with a boiling point at normal pressure above about 200° C. are understood to mean non-volatile siloxanes. Preferred siloxanes are polydialkylsiloxanes, such as polydimethylsiloxane, polyalkylarylsiloxanes, such as polyphenylmethylsiloxane, ethoxylated polydialkylsiloxanes and polydialkylsiloxanes which contain amine and/or hydroxy groups.

In particular the polyvinylpyrrolidones and the vinylpyrrolidone/vinyl acetate copolymers are preferred as contemplated herein.

A second group of particularly preferred polymers is constituted by the amphoteric polymers. The use of a copolymer of i) N-tert-octylacrylamide, ii) acrylic acid, iii) tert.-butylaminoethyl methacrylate and optionally further monomers is very particularly preferred.

Preferred amphoteric copolymers preferably include to an extent of at least about 90% by weight, preferentially to an extent of at least about 95% by weight, and in particular to an extent of at least about 97% by weight of the monomers N-tert-octylacrylamide, acrylic acid and tert.-butylaminoethyl methacrylate. Corresponding copolymers can also be obtained with use exclusively of the monomers N-tert-octylacrylamide, acrylic acid and tert.-butylaminoethyl methacrylate.

Copolymers formed form the monomers N-tert-octylacrylamide, acrylic acid, tert.-butylaminoethyl methacrylate, methyl methacrylate and hydroxypropyl methacrylate are also preferred. It is particularly preferred in particular if the copolymer includes to an extent of at least about 90% by weight, preferentially to an extent of at least about 95% by weight, and in particular to an extent of at least about 97% by weight of the monomers N-tert-octylacrylamide, acrylic acid, tert.-butylaminoethyl methacrylate, methyl methacrylate and hydroxypropyl methacrylate.

The above-described amphoteric copolymers are sold for example under the name Amphomer® (INCI name: Octylacrylamide/Acrylates/Butylaminoethyl Methacrylate Copolymer; CAS number 70801-07-9) by the company National Starch.

A fourth essential constituent of the cosmetic compositions is the propellant. It has proven to be particularly advantageous from a cosmetic and technical viewpoint if the weight share of the propellant in the total weight of the composition is from about 25 to about 50% by weight, preferably from about 30 to about 50% by weight, and in particular from about 35 to about 45% by weight.

Preferred propellants (propellant gases) are selected from propane, propene, n-butane, iso-butane, iso-butene, n-pentane, pentene, iso-pentane, iso-pentene, methane, ethane, dimethylether, nitrogen, air, oxygen, nitrous oxide, dichlorofluoromethane, chlorodifluoromethane, chlorofluoromethane, 1,1,2,2-tetrachloro-1-fluoroethane, 1,1,1,2-tetrachloro-2-fluoroethane, 1,2,2-trichloro-1,1-difluoroethane, 1,1,2-trichloro-1,2-difluoroethane, 1,1,1-trichloro-2,2-difluoroethane, 2,2-dichloro-1,1,1-trifluoroethane, 1,2-dichloro-1,1,2-trifluoroethane, 2-chloro-1,1,1,2-tetrafluoroethane, 1-chloro-1,1,2,2-tetrafluoroethane, 1,1,2-trichloro-2-fluoroethane, 1,2-dichloro-1,2-difluoroethane, 1,2-dichloro-1,1-difluoroethane, 1-chloro-1,2,2-trifluoroethane, 2-chloro-1,1,1-trifluoroethane, 1-chloro-1,1,2-trifluoroethane, 1,2-dichloro-1-fluoroethane, 1,1-dichloro-1-fluoroethane, 2-chloro-1,1-difluoroethane, 1-chloro-1,1-difluoroethane, 1-chloro-2-fluoro ethane, 1-chloro-1-fluorethane, 2-chloro-1,1-difluoroethene, 1,1,1,3-tetrafluoro ethane, heptafluoro-n-propane, perfluoroethane, monochlorodifluoromethane, 1,1-difluoroethane, more specifically either individually or in combination.

The use of propane, propane/butane mixtures or dimethyl ether is preferred, particularly preferably the use of dimethyl ether.

The composition of some preferred cosmetic compositions can be deduced from the following overview 1 (values in % by weight relate to the total weight of the cosmetic composition).

| | Formula 1 | Formula 2 | Formula 3 | Formula 4 | Formula 5 |
|---|---|---|---|---|---|
| Fibers | 0.1 to 5.0 | 0.1 to 2.0 | 0.1 to 2.0 | 0.1 to 1.0 | 0.1 to 0.5 |
| Ethanol | 20 to 50 | 20 to 50 | 20 to 45 | 25 to 40 | 25 to 40 |
| Polymer | 0.5 to 8.0 | 0.5 to 8.0 | 1.0 to 6.0 | 1.0 to 6.0 | 2.0 to 4.0 |
| Propellant | 30 to 70 | 35 to 65 | 35 to 65 | 40 to 60 | 40 to 60 |
| Optional additives | to 100 | to 100 | to 100 | to 100 | to 100 |

| | Formula 1a | Formula 2a | Formula 3a | Formula 4a | Formula 5a |
|---|---|---|---|---|---|
| Fibers | 0.1 to 5.0 | 0.1 to 2.0 | 0.1 to 2.0 | 0.1 to 1.0 | 0.1 to 0.5 |
| Ethanol | 20 to 50 | 20 to 50 | 20 to 45 | 25 to 40 | 25 to 40 |
| Polyvinylpyrrolidone, | 0.5 to 8.0 | 0.5 to 8.0 | 1.0 to 6.0 | 1.0 to 6.0 | 2.0 to 4.0 |
| vinylpyrrolidone/vinyl | | | | | |
| acetate copolymer | | | | | |
| Propellant | 30 to 70 | 35 to 65 | 35 to 65 | 40 to 60 | 40 to 60 |
| Optional additives | to 100 | to 100 | to 100 | to 100 | to 100 |

| | Formula 1b | Formula 2b | Formula 3b | Formula 4b | Formula 5b |
|---|---|---|---|---|---|
| Fibers | 0.1 to 5.0 | 0.1 to 2.0 | 0.1 to 2.0 | 0.1 to 1.0 | 0.1 to 0.5 |
| Ethanol | 20 to 50 | 20 to 50 | 20 to 45 | 25 to 40 | 25 to 40 |
| N-tert-octylacrylamide, | 0.5 to 8.0 | 0.5 to 8.0 | 1.0 to 6.0 | 1.0 to 6.0 | 2.0 to 4.0 |
| acrylic acid, tert.- | | | | | |
| butylaminoethyl | | | | | |
| methacrylate copolymer | | | | | |
| Propellant | 30 to 70 | 35 to 65 | 35 to 65 | 40 to 60 | 40 to 60 |
| Optional additives | to 100 | to 100 | to 100 | to 100 | to 100 |

| | Formula 6 | Formula 7 | Formula 8 | Formula 9 | Formula 10 |
|---|---|---|---|---|---|
| Cellulose fibers | 0.1 to 5.0 | 0.1 to 2.0 | 0.1 to 2.0 | 0.1 to 1.0 | 0.1 to 0.5 |
| Ethanol | 20 to 50 | 20 to 50 | 20 to 45 | 25 to 40 | 25 to 40 |
| Polymer | 0.5 to 8.0 | 0.5 to 8.0 | 1.0 to 6.0 | 1.0 to 6.0 | 2.0 to 4.0 |
| Propellant | 30 to 70 | 35 to 65 | 35 to 65 | 40 to 60 | 40 to 60 |
| Optional additives | to 100 | to 100 | to 100 | to 100 | to 100 |

| | Formula 6a | Formula 7a | Formula 8a | Formula 9a | Formula 10a |
|---|---|---|---|---|---|
| Cellulose fibers | 0.1 to 5.0 | 0.1 to 2.0 | 0.1 to 2.0 | 0.1 to 1.0 | 0.1 to 0.5 |
| Ethanol | 20 to 50 | 20 to 50 | 20 to 45 | 25 to 40 | 25 to 40 |
| Polyvinylpyrrolidone, | 0.5 to 8.0 | 0.5 to 8.0 | 1.0 to 6.0 | 1.0 to 6.0 | 2.0 to 4.0 |
| vinylpyrrolidone/vinyl | | | | | |
| acetate copolymer | | | | | |
| Propellant | 30 to 70 | 35 to 65 | 35 to 65 | 40 to 60 | 40 to 60 |
| Optional additives | to 100 | to 100 | to 100 | to 100 | to 100 |

| | Formula 6b | Formula 7b | Formula 8b | Formula 9b | Formula 10b |
|---|---|---|---|---|---|
| Cellulose fibers | 0.1 to 5.0 | 0.1 to 2.0 | 0.1 to 2.0 | 0.1 to 1.0 | 0.1 to 0.5 |
| Ethanol | 20 to 50 | 20 to 50 | 20 to 45 | 25 to 40 | 25 to 40 |
| N-tert-octylacrylamide, | 0.5 to 8.0 | 0.5 to 8.0 | 1.0 to 6.0 | 1.0 to 6.0 | 2.0 to 4.0 |
| acrylic acid, tert.- | | | | | |
| butylaminoethyl | | | | | |
| methacrylate copolymer | | | | | |
| Propellant | 30 to 70 | 35 to 65 | 35 to 65 | 40 to 60 | 40 to 60 |
| Optional additives | to 100 | to 100 | to 100 | to 100 | to 100 |

| | Formula 11 | Formula 12 | Formula 13 | Formula 14 | Formula 15 |
|---|---|---|---|---|---|
| Fibers with a fiber | 0.1 to 5.0 | 0.1 to 2.0 | 0.1 to 2.0 | 0.1 to 1.0 | 0.1 to 0.5 |
| length between 1.0 | | | | | |
| and 200 μm | | | | | |
| Ethanol | 20 to 50 | 20 to 50 | 20 to 45 | 25 to 40 | 25 to 40 |
| Polymer | 0.5 to 8.0 | 0.5 to 8.0 | 1.0 to 6.0 | 1.0 to 6.0 | 2.0 to 4.0 |
| Propellant | 30 to 70 | 35 to 65 | 35 to 65 | 40 to 60 | 40 to 60 |
| Optional additives | to 100 | to 100 | to 100 | to 100 | to 100 |

| | Formula 11a | Formula 12a | Formula 13a | Formula 14a | Formula 15a |
|---|---|---|---|---|---|
| Fibers with a fiber | 0.1 to 5.0 | 0.1 to 2.0 | 0.1 to 2.0 | 0.1 to 1.0 | 0.1 to 0.5 |
| length between 1.0 | | | | | |
| and 200 μm | | | | | |
| Ethanol | 20 to 50 | 20 to 50 | 20 to 45 | 25 to 40 | 25 to 40 |
| Polyvinylpyrrolidone, | 0.5 to 8.0 | 0.5 to 8.0 | 1.0 to 6.0 | 1.0 to 6.0 | 2.0 to 4.0 |
| vinylpyrrolidone/vinyl | | | | | |
| acetate copolymer | | | | | |
| Propellant | 30 to 70 | 35 to 65 | 35 to 65 | 40 to 60 | 40 to 60 |
| Optional additives | to 100 | to 100 | to 100 | to 100 | to 100 |

| | Formula 11b | Formula 12b | Formula 13b | Formula 14b | Formula 15b |
|---|---|---|---|---|---|
| Fibers with a fiber | 0.1 to 5.0 | 0.1 to 2.0 | 0.1 to 2.0 | 0.1 to 1.0 | 0.1 to 0.5 |
| length between 1.0 | | | | | |
| and 200 μm | | | | | |
| Ethanol | 20 to 50 | 20 to 50 | 20 to 45 | 25 to 40 | 25 to 40 |
| N-tert-octylacrylamide, | 0.5 to 8.0 | 0.5 to 8.0 | 1.0 to 6.0 | 1.0 to 6.0 | 2.0 to 4.0 |
| acrylic acid, tert.- | | | | | |
| butylaminoethyl | | | | | |
| methacrylate copolymer | | | | | |
| Propellant | 30 to 70 | 35 to 65 | 35 to 65 | 40 to 60 | 40 to 60 |
| Optional additives | to 100 | to 100 | to 100 | to 100 | to 100 |

| | Formula 16 | Formula 17 | Formula 18 | Formula 19 | Formula 20 |
|---|---|---|---|---|---|
| Cellulose fibers with a | 0.1 to 5.0 | 0.1 to 2.0 | 0.1 to 2.0 | 0.1 to 1.0 | 0.1 to 0.5 |
| fiber length between | | | | | |
| 1.0 and 200 μm | | | | | |
| Ethanol | 20 to 50 | 20 to 50 | 20 to 45 | 25 to 40 | 25 to 40 |
| Polymer | 0.5 to 8.0 | 0.5 to 8.0 | 1.0 to 6.0 | 1.0 to 6.0 | 2.0 to 4.0 |
| Propellant | 30 to 70 | 35 to 65 | 35 to 65 | 40 to 60 | 40 to 60 |
| Optional additives | to 100 | to 100 | to 100 | to 100 | to 100 |
| | Formula 16a | Formula 17a | Formula 18a | Formula 19 | Formula 20a |
| Cellulose fibers with a | 0.1 to 5.0 | 0.1 to 2.0 | 0.1 to 2.0 | 0.1 to 1.0 | 0.1 to 0.5 |
| fiber length between | | | | | |
| 1.0 and 200 μm | | | | | |
| Ethanol | 20 to 50 | 20 to 50 | 20 to 45 | 25 to 40 | 25 to 40 |
| Polyvinylpyrrolidone, | 0.5 to 8.0 | 0.5 to 8.0 | 1.0 to 6.0 | 1.0 to 6.0 | 2.0 to 4.0 |
| vinylpyrrolidone/vinyl | | | | | |
| acetate copolymer | | | | | |
| Propellant | 30 to 70 | 35 to 65 | 35 to 65 | 40 to 60 | 40 to 60 |
| Optional additives | to 100 | to 100 | to 100 | to 100 | to 100 |

| | Formula 16b | Formula 17b | Formula 18b | Formula 19b | Formula 20b |
|---|---|---|---|---|---|
| Cellulose fibers with a | 0.1 to 5.0 | 0.1 to 2.0 | 0.1 to 2.0 | 0.1 to 1.0 | 0.1 to 0.5 |
| fiber length between | | | | | |
| 1.0 and 200 μm | | | | | |
| Ethanol | 20 to 50 | 20 to 50 | 20 to 45 | 25 to 40 | 25 to 40 |
| N-tert-octylacrylamide, | 0.5 to 8.0 | 0.5 to 8.0 | 1.0 to 6.0 | 1.0 to 6.0 | 2.0 to 4.0 |
| acrylic acid, tert.- | | | | | |
| butylaminoethyl | | | | | |
| methacrylate copolymer | | | | | |
| Propellant | 30 to 70 | 35 to 65 | 35 to 65 | 40 to 60 | 40 to 60 |
| Optional additives | to 100 | to 100 | to 100 | to 100 | to 100 |

| | Formula 21 | Formula 22 | Formula 23 | Formula 24 | Formula 25 |
|---|---|---|---|---|---|
| Fibers | 0.1 to 5.0 | 0.1 to 2.0 | 0.1 to 2.0 | 0.1 to 1.0 | 0.1 to 0.5 |
| Ethanol | 20 to 50 | 20 to 50 | 20 to 45 | 25 to 40 | 25 to 40 |
| Polymer | 0.5 to 8.0 | 0.5 to 8.0 | 1.0 to 6.0 | 1.0 to 6.0 | 2.0 to 4.0 |
| Dimethyl ether | 30 to 70 | 35 to 65 | 35 to 65 | 40 to 60 | 40 to 60 |
| Optional additives | to 100 | to 100 | to 100 | to 100 | to 100 |

| | Formula 21a | Formula 22a | Formula 23a | Formula 24a | Formula 25a |
|---|---|---|---|---|---|
| Fibers | 0.1 to 5.0 | 0.1 to 2.0 | 0.1 to 2.0 | 0.1 to 1.0 | 0.1 to 0.5 |
| Ethanol | 20 to 50 | 20 to 50 | 20 to 45 | 25 to 40 | 25 to 40 |
| Polyvinylpyrrolidone, | 0.5 to 8.0 | 0.5 to 8.0 | 1.0 to 6.0 | 1.0 to 6.0 | 2.0 to 4.0 |
| vinylpyrrolidone/vinyl | | | | | |
| acetate copolymer | | | | | |
| Dimethyl ether | 30 to 70 | 35 to 65 | 35 to 65 | 40 to 60 | 40 to 60 |
| Optional additives | to 100 | to 100 | to 100 | to 100 | to 100 |

| | Formula 21b | Formula 22b | Formula 23b | Formula 24b | Formula 25b |
|---|---|---|---|---|---|
| Fibers | 0.1 to 5.0 | 0.1 to 2.0 | 0.1 to 2.0 | 0.1 to 1.0 | 0.1 to 0.5 |
| Ethanol | 20 to 50 | 20 to 50 | 20 to 45 | 25 to 40 | 25 to 40 |
| N-tert-octylacrylamide, | 0.5 to 8.0 | 0.5 to 8.0 | 1.0 to 6.0 | 1.0 to 6.0 | 2.0 to 4.0 |
| acrylic acid, tert.- | | | | | |
| butylaminoethyl | | | | | |
| methacrylate copolymer | | | | | |
| Dimethyl ether | 30 to 70 | 35 to 65 | 35 to 65 | 40 to 60 | 40 to 60 |
| Optional additives | to 100 | to 100 | to 100 | to 100 | to 100 |

| | Formula 26 | Formula 27 | Formula 28 | Formula 29 | Formula 30 |
|---|---|---|---|---|---|
| Cellulose fibers | 0.1 to 5.0 | 0.1 to 2.0 | 0.1 to 2.0 | 0.1 to 1.0 | 0.1 to 0.5 |
| Ethanol | 20 to 50 | 20 to 50 | 20 to 45 | 25 to 40 | 25 to 40 |
| Polymer | 0.5 to 8.0 | 0.5 to 8.0 | 1.0 to 6.0 | 1.0 to 6.0 | 2.0 to 4.0 |
| Dimethyl ether | 30 to 70 | 35 to 65 | 35 to 65 | 40 to 60 | 40 to 60 |
| Optional additives | to 100 | to 100 | to 100 | to 100 | to 100 |

| | Formula 26a | Formula 27a | Formula 28a | Formula 29a | Formula 30a |
|---|---|---|---|---|---|
| Cellulose fibers | 0.1 to 5.0 | 0.1 to 2.0 | 0.1 to 2.0 | 0.1 to 1.0 | 0.1 to 0.5 |
| Ethanol | 20 to 50 | 20 to 50 | 20 to 45 | 25 to 40 | 25 to 40 |
| Polyvinylpyrrolidone, | 0.5 to 8.0 | 0.5 to 8.0 | 1.0 to 6.0 | 1.0 to 6.0 | 2.0 to 4.0 |
| vinylpyrrolidone/vinyl | | | | | |
| acetate copolymer | | | | | |
| Dimethyl ether | 30 to 70 | 35 to 65 | 35 to 65 | 40 to 60 | 40 to 60 |
| Optional additives | to 100 | to 100 | to 100 | to 100 | to 100 |

| | Formula 26b | Formula 27b | Formula 28b | Formula 29b | Formula 30b |
|---|---|---|---|---|---|
| Cellulose fibers | 0.1 to 5.0 | 0.1 to 2.0 | 0.1 to 2.0 | 0.1 to 1.0 | 0.1 to 0.5 |
| Ethanol | 20 to 50 | 20 to 50 | 20 to 45 | 25 to 40 | 25 to 40 |
| N-tert-octylacrylamide, | 0.5 to 8.0 | 0.5 to 8.0 | 1.0 to 6.0 | 1.0 to 6.0 | 2.0 to 4.0 |
| acrylic acid, tert.- | | | | | |
| butylaminoethyl | | | | | |
| methacrylate copolymer | | | | | |
| Dimethyl ether | 30 to 70 | 35 to 65 | 35 to 65 | 40 to 60 | 40 to 60 |
| Optional additives | to 100 | to 100 | to 100 | to 100 | to 100 |

| | Formula 31 | Formula 32 | Formula 33 | Formula 34 | Formula 35 |
|---|---|---|---|---|---|
| Fibers with a fiber | 0.1 to 5.0 | 0.1 to 2.0 | 0.1 to 2.0 | 0.1 to 1.0 | 0.1 to 0.5 |
| length between 1.0 | | | | | |
| and 200 μm | | | | | |
| Ethanol | 20 to 50 | 20 to 50 | 20 to 45 | 25 to 40 | 25 to 40 |
| Polymer | 0.5 to 8.0 | 0.5 to 8.0 | 1.0 to 6.0 | 1.0 to 6.0 | 2.0 to 4.0 |
| Dimethyl ether | 30 to 70 | 35 to 65 | 35 to 65 | 40 to 60 | 40 to 60 |
| Optional additives | to 100 | to 100 | to 100 | to 100 | to 100 |

| | Formula 31a | Formula 32a | Formula 33a | Formula 34a | Formula 35a |
|---|---|---|---|---|---|
| Fibers with a fiber | 0.1 to 5.0 | 0.1 to 2.0 | 0.1 to 2.0 | 0.1 to 1.0 | 0.1 to 0.5 |
| length between 1.0 | | | | | |
| and 200 μm | | | | | |
| Ethanol | 20 to 50 | 20 to 50 | 20 to 45 | 25 to 40 | 25 to 40 |
| Polyvinylpyrrolidone, | 0.5 to 8.0 | 0.5 to 8.0 | 1.0 to 6.0 | 1.0 to 6.0 | 2.0 to 4.0 |
| vinylpyrrolidone/vinyl | | | | | |
| acetate copolymer | | | | | |
| Dimethyl ether | 30 to 70 | 35 to 65 | 35 to 65 | 40 to 60 | 40 to 60 |
| Optional additives | to 100 | to 100 | to 100 | to 100 | to 100 |

| | Formula 31b | Formula 32b | Formula 33b | Formula 34b | Formula 35b |
|---|---|---|---|---|---|
| Fibers with a fiber | 0.1 to 5.0 | 0.1 to 2.0 | 0.1 to 2.0 | 0.1 to 1.0 | 0.1 to 0.5 |
| length between 1.0 | | | | | |
| and 200 μm | | | | | |
| Ethanol | 20 to 50 | 20 to 50 | 20 to 45 | 25 to 40 | 25 to 40 |
| N-tert-octylacrylamide, | 0.5 to 8.0 | 0.5 to 8.0 | 1.0 to 6.0 | 1.0 to 6.0 | 2.0 to 4.0 |
| acrylic acid, tert.- | | | | | |
| butylaminoethyl | | | | | |
| methacrylate copolymer | | | | | |
| Dimethyl ether | 30 to 70 | 35 to 65 | 35 to 65 | 40 to 60 | 40 to 60 |
| Optional additives | to 100 | to 100 | to 100 | to 100 | to 100 |

| | Formula 36 | Formula 37 | Formula 38 | Formula 39 | Formula 40 |
|---|---|---|---|---|---|
| Cellulose fibers with a | 0.1 to 5.0 | 0.1 to 2.0 | 0.1 to 2.0 | 0.1 to 1.0 | 0.1 to 0.5 |
| fiber length between | | | | | |
| 1.0 and 200 μm | | | | | |
| Ethanol | 20 to 50 | 20 to 50 | 20 to 45 | 25 to 40 | 25 to 40 |
| Polymer | 0.5 to 8.0 | 0.5 to 8.0 | 1.0 to 6.0 | 1.0 to 6.0 | 2.0 to 4.0 |
| Dimethyl ether | 30 to 70 | 35 to 65 | 35 to 65 | 40 to 60 | 40 to 60 |
| Optional additives | to 100 | to 100 | to 100 | to 100 | to 100 |
| | Formula 36a | Formula 37a | Formula 38a | Formula 39a | Formula 40a |
| Cellulose fibers with a | 0.1 to 5.0 | 0.1 to 2.0 | 0.1 to 2.0 | 0.1 to 1.0 | 0.1 to 0.5 |
| fiber length between | | | | | |
| 1.0 and 200 μm | | | | | |
| Ethanol | 20 to 50 | 20 to 50 | 20 to 45 | 25 to 40 | 25 to 40 |
| Polyvinylpyrrolidone, | 0.5 to 8.0 | 0.5 to 8.0 | 1.0 to 6.0 | 1.0 to 6.0 | 2.0 to 4.0 |
| vinylpyrrolidone/vinyl | | | | | |
| acetate copolymer | | | | | |
| Dimethyl ether | 30 to 70 | 35 to 65 | 35 to 65 | 40 to 60 | 40 to 60 |
| Optional additives | to 100 | to 100 | to 100 | to 100 | to 100 |

| | Formula 36b | Formula 37b | Formula 38b | Formula 39b | Formula 40b |
|---|---|---|---|---|---|
| Cellulose fibers with a | 0.1 to 5.0 | 0.1 to 2.0 | 0.1 to 2.0 | 0.1 to 1.0 | 0.1 to 0.5 |
| fiber length between | | | | | |
| 1.0 and 200 μm | | | | | |
| Ethanol | 20 to 50 | 20 to 50 | 20 to 45 | 25 to 40 | 25 to 40 |
| N-tert-octylacrylamide, | 0.5 to 8.0 | 0.5 to 8.0 | 1.0 to 6.0 | 1.0 to 6.0 | 2.0 to 4.0 |
| acrylic acid, tert.- | | | | | |
| butylaminoethyl | | | | | |
| methacrylate copolymer | | | | | |
| Dimethyl ether | 30 to 70 | 35 to 65 | 35 to 65 | 40 to 60 | 40 to 60 |
| Optional additives | to 100 | to 100 | to 100 | to 100 | to 100 |

Particularly preferred cosmetic compositions also contain
(e) from about 0 to about 10% by weight of dye(s)
(f) from about 0 to about 10% by weight of pigment(s)
   with the provision that
   the sum of the amounts of ingredients (e) and (f) is from about 0.0001 to about 10% by weight, in relation to the total weight of the cosmetic composition.

In other words, the compositions can contain 0% by weight of dyes (in that case at least one pigment must be present in an amount of at least about 0.0001% by weight), the dye content however can also be about 10% by weight (in that case, pigment can no longer be present). Regardless of whether
only one dye,
a plurality of dyes,
only one pigment,
a plurality of pigments,
one dye and a plurality of pigments
a plurality of dyes and one pigment
a plurality of dyes and a plurality of pigments
   is/are used, the total amount of all dyes and pigments (also referred to as total amount of color-giving components) in the cosmetic composition is from about 0.0001 to about 10% by weight, in relation to the total weight of the cosmetic composition.

It has been found that such colored cosmetic compositions and/or those that have been mixed with dyes or pigments not only increase hair volume and increase the fullness and give a feel of “having more hair”, but the coloring of the products can at the same time cover bald spots without the color rubbing off on the hands or clothing or pillows.

Preferred cosmetic compositions contain from about 0.0001 to about 10% by weight, preferably from about 0.01 to about 9.5% by weight, more preferably from about 0.05 to about 9% by weight, even more preferably from about 0.1 to about 8.5% by weight, and in particular from about 0.25 to about 8% by weight of dye(s), in each case in relation to the total weight of the cosmetic composition.

Dyes are colorants, i.e. color-giving substances, which in contrast to pigments are soluble in the application medium (for example in water, oils or other solvents).

A dye or a mixture of dyes can be selected depending on the desired color, color intensity and fastness properties of the resultant coloring. All dyes that are conventional and commercially obtainable in the field of cosmetics can be used without difficulty within the scope of the present disclosure.

Cosmetic compositions that are likewise preferred contain from about 0.0001 to about 10% by weight, preferably from about 0.01 to about 9.5% by weight, more preferably from about 0.05 to about 9% by weight, even more preferably from about 0.1 to about 8.5% by weight, and in particular from about 0.25 to about 8% by weight of pigment(s), in each case in relation to the total weight of the cosmetic composition.

All types of water-insoluble pigments, for example natural inorganic pigments (also referred to as mineral pigments) are suitable in principle. These pigments contain predominantly sulfides and oxides. Examples of such pigments are ocher (Fe(OOH); Pigment Yellow 43), burnt sienna (Fe₂O₃; Pigment Red 102), umbra (Fe₂O₃×MnO₂; Pigment Brown 7:x), cinnabar (β-HgS, PR 106), lapis lazuli (ultramarine, Na₆Al₆Si₆O₂₄×Na₂Sn; Pigment Blue 29), azurite (basic copper carbonate, Cu₃[OH/CO₃]₂; PB 30), green earth (FeO-containing silicate; Pigment Green 23), malachite (Cu₂[(OH)₂, CO₃]) and carbon black (carbon (graphite), Pigment Black 9). With regard to the avoidance of undesirable visible residues or gray films or the water-resistant temporary coloring of the fibers, however, the use of synthetic inorganic pigments has proven to be advantageous. Synthetic inorganic pigments are produced for example by chemical and/or physical conversion (digestion, precipitation, annealing). They include, in particular,

white pigments (titanium dioxide (TiO₂), Pigment White PW 6; zinc sulfide (ZnS), PW 7; zinc oxide (ZnO), PW 4; antimony white (Sb₂O₃), PW 11; lithopone (ZnS/BaSO₄), PW 5; white lead (2PbCO₃×Pb(OH)₂), PW 1),

subordinate white fillers (calcium carbonate, PW 18; talc, PW 26 and barium sulfate, PW 21);

black pigments (manganese black, spinel black and carbon black pigments (graphite carbon);

luster pigments (absorption pigments, metal pigments or metal effect pigments and pearlescent pigments) and

inorganic colored pigments (iron oxide pigments, iron blue pigments, ultramarine pigments and, although less preferred on account of their toxicological properties, lead chromate pigments, chromium oxide pigments, cadmium pigments and bismuth vanadate pigments).

Preferred synthetic inorganic pigments are metal pigments or metal effect pigments formed from powdered metals or metal alloys, such as aluminum bronzes (metal: Al) gold bronzes (metal: Cu, Cu—Al or Cu—Zn alloy), silver bronzes (metal: Cu—Zn—Ni), flame-colored bronzes (metal: oxidized Cu—Zn) and patent bronzes (metal: Cu—Zn—(Ni)+dye).

Further preferred synthetic inorganic pigments are pearlescent pigments which include a number of layers with different refractive index. Examples of such pearlescent pigments are magnesium stearate, zinc stearate and lithium stearate or ethylene glycol distearate or polyethylene terephthalate and pearlescent pigments which consist substantially of mica, titanium dioxide (titanium dioxide mica), bismuth chloride oxide or guanine, and additionally can be coated with colored oxide layers (for example iron oxides or chromium oxides). Mica-based and mica/metal oxide-based pearlescent pigments are particularly preferred pearlescent pigments as contemplated herein. Micas are sheet silicates. The most important representatives of these silicates are muscovite, phlogopite, paragonite, biotite, lepidolite and margarite. In order to produce the pearlescent pigments in conjunction with metal oxides, the mica, predominantly muscovite or phlogopite, is coated with a metal oxide. Suitable metal oxides are, amongst others, TiO2, Cr2O3 and Fe2O3. Interference pigments and colored luster pigments are obtained by corresponding coating as pearlescent pigments preferred as contemplated herein. These pearlescent pigment types additionally have color effects besides a sparkly optical effect. Furthermore, the pearlescent pigments usable as contemplated herein can additionally contain a color pigment which does not derive from a metal oxide.

Very particularly preferred pearlescent pigments are pigments that are marketed by the company Merck under the trade names Colorona®, wherein the pigments Colorona® red-brown (47-57% by weight Muscovit Mica (KH₂(AlSiO₄)₃), 43-50% by weight Fe₂O₃ (INCI: Iron Oxides CI 77491), <3% by weight TiO₂ (INCI: Titanium Dioxide CI 77891), Colorona® Blackstar Blue (39-47% by weight Muscovit Mica (KH₂(AlSiO₄)₃), 53-61% by weight Fe₃O₄ (INCI: Iron Oxides CI 77499)), Colorona® Siena Fine (35-45% by weight Muscovit Mica (KH₂(AlSiO₄)₃), 55-65% by weight Fe₂O₃ (INCI: Iron Oxides CI 77491)), Colorona® Aborigine Amber (50-62% by weight Muscovit Mica (KH₂(AlSiO₄)₃), 36-44% by weight Fe₃O₄ (INCI: Iron Oxides CI 77499), 2-6% by weight TiO₂ (INCI: Titanium Dioxide CI 77891)), Colorona® Patagonian Purple (42-54% by weight Muscovit Mica (KH₂(AlSiO₄)₃), 26-32% by weight Fe₂O₃ (INCI: Iron Oxides CI 77491), 18-22% by weight TiO₂ (INCI: Titanium Dioxide CI 77891), 2-4% by weight Prussian Blue (INCI: Ferric Ferrocyanide CI 77510)), Colorona® Chameleon (40-50% by weight Muscovit Mica (KH₂(AlSiO₄)₃), 50-60% by weight Fe₂O₃ (INCI: Iron Oxides CI 77491)) and Silk® Mica (>98% by weight Muscovit Mica (KH₂(AlSiO₄)₃)) are particularly preferred.

A group of particularly preferred pigments is formed by the color-giving synthetic iron oxides. Particularly preferred representatives of this substance class are Pigment Brown 6 (CI No 77491), Pigment Red 101 (CI No 77491), Pigment Yellow 42 (CI No 77492), Pigment Black 11 (CI No 77499) and mixtures of these pigments.

Pigments that can be used very particularly preferably are exemplified by a good spray ability in the compositions as contemplated herein, a homogeneous spray pattern, and an extremely low tendency to block the spray nozzles (“clogging”). Cosmetic compositions that are extremely preferred as contemplated herein are exemplified in that they contain at least one pigment from the group CI12490, CI14700, CI14720, CI15510, CI15985, CI45380, CI47005, CI60730, CI61565, CI73360, CI74160, CI77007, CI77019, CI77288, CI77289, CI77491, CI77492, wherein the total amount of pigment(s) from this group is from about 0.0001 to about 10% by weight, preferably from about 0.01 to about 9.5% by weight, more preferably from about 0.05 to about 9% by weight, even more preferably from about 0.1 to about 8.5% by weight, and in particular from about 0.25 to about 8% by weight.

The cosmetic compositions particularly preferably contain at least one starch compound. This starch compound can be the only polymer in the products as contemplated herein, however it is preferred to use the starch compound in addition to the preferably film-forming polymers described further above. The wording “as further constituent” therefore means “additionally to” all other previously mentioned obligatorily necessary constituents.

Preferred cosmetic compositions contain, as further constituent, from about 1.0 to about 10% by weight, preferably from about 2.0 to about 6.0% by weight, and in particular from about 3.0 to about 5.0% by weight of at least one starch compound.

Starch is a reserve carbohydrate which is stored by many plants in the form of starch grains (granules) usually from about 1 to about 200 μm in size in different plant parts, for example in tubers or roots, grain seeds, fruits or in the pith. Starch belongs to the family of homoglycans and is a polycondensation product of D-glucose. Starches that are preferred as contemplated herein are selected from at least one polycondensation product of D-glucose obtained from natural and/or physically modified starch of potatoes, corn, rice, peas, acorns, chestnuts, barley, wheat, bananas, sago, millet, sorghum, oat, rye, beans, batate, maranta or manioc and mixtures thereof. With regard to cleaning power, the use of native and/or physically modified rice starch has proven to be particularly advantageous. Native starch is understood to mean a starch that is isolated from starch-containing plants and which, after the isolation and purification, was neither physically nor chemically modified. By contrast, physically modified starch is understood to mean a starch that was subjected to at least one physical modification after the isolation. Here, “physical modification” is understood to mean modification with application of pressure and/or heat and/or light. A modification by employing chemical and enzyme reactions, for example the hydrolysis of the starch, however, does not fall under the term “physical modification”. A preferably used physical modification is the application of heat, in particular the boiling of the natural starch. Preferred embodiments of the present disclosure compositions contain at least one starch selected from chemically and/or physically modified rice starches, in particular from physically modified rice starch. As a result of the use of native and/or physically modified rice starches in combination with dyes and/or pigments, a particularly high adhesion of the dyes and/or pigments to the starch is attained, and therefore, besides the excellent cleaning power, a long-lasting temporary hair coloring is also made possible.

It is hereby possible and preferred to use pre-colored and/or pre-pigmented starches, which can be sprayed excellently with the cosmetic compositions. Particularly preferred cosmetic compositions contain ingredients (e) and/or (f) in the form of a starch-dye compound and/or starch-pigment compound, preferably in the form of dyed and/or pigmented rice starch.

It has additionally been found that the composition of the starch particles itself has proven to be relevant for the cosmetic effect. Preferably used particles therefore contain a certain proportion of the native and/or physically modified starch, in particular rice starch. It is thus preferred if the starch-dye compound contains at least one starch, in particular a physically modified rice starch, in a total amount of from about 70 to about 96% by weight, in particular from about 80 to about 94% by weight, in each case in relation to the total weight of the particle (compound). The use of particles which contain a higher weight share of physically modified rice starch leads to particularly homogeneous spray patterns and preferred adhesion of the color to the keratin fibers.

In addition to such compounds formed from starch and color-giving substances or instead of them, the fibers of the cosmetic compositions can of course also be pre-colored and/or pre-pigmented. Here, particularly preferred cosmetic compositions are those which contain the ingredients (a) and (e) and/or (a) and (f) in the form of a fiber-dye compound or a fiber-pigment compound, preferably in the form of dyed and/or pigmented fibers.

Preferred cosmetic compositions contain at least one ester oil as further constituent. The group of ester oils includes in particular esters of C₆-C₃₀ fatty acids with C₂-C₃₀ fatty alcohols, preferably monoesters of fatty acids with alcohols with 2 to 24 C atoms. Exemplary ester oils are isopropyl myristate (Rilanit® IPM), isononanoic acid-C16-18-alkyl ester (Cetiol® SN), 2-ethylhexyl palmitate (Cegesoft® 24), stearic acid-2-ethylhexyl ester (Cetiol® 868), cetyloleate, glycerine tricaprylate, coconut fatty alcohol caprinate/caprylate (Cetiol® LC), n-butylstearate, oleylerucate (Cetiol® J 600), isopropyl palmitate (Rilanit® IPP), oleyl oleate (Cetiol®), lauric acid hexyl ester (Cetiol® A), di-n-butyl adipate (Cetiol® B), myristyl myristate (Cetiol® MM), cetearyl isononanoate (Cetiol® SN) and oleic acid decyl ester (Cetiol® V). The use of isopropyl myristate is particularly preferred.

The weight share of the ester oil in the total weight of the cosmetic composition is preferentially from about 0.01 to about 1.0% by weight, preferably from about 0.02 to about 0.6% by weight, and in particular from about 0.03 to about 0.2% by weight.

The fibers described at the outset as first essential constituent in the cosmetic compositions can be modified differently. Such modifications relate for example to the length of the fibers described further above or the length-to-diameter ratio thereof.

A further possibility for influencing the technical and cosmetic properties of the compositions lies in the modification of the surface properties of the used fibers.

In a first preferred embodiment, hydrophobized fibers are used. The hydrophobization (impregnation) provides the fibers with a water-repelling property. For example, paraffins or waxes, but also film-forming silicones are suitable as hydrophobizing agents.

Depending on the intended purpose of the cosmetic composition, it can be advantageous if the fibers have a certain oil absorption capacity. This oil absorption capacity can be influenced by employing the previously described hydrophobization of the fibers. The use of hydrophobized or non-hydrophobized fibers with an oil absorption capacity above about 0.2 g jojoba oil per gram of fibers is preferred. The use of fibers with an oil absorption capacity (20° C.) of from about 0.2 to about 0.8 g jojoba oil/g, preferably from about 0.3 to about 0.7 g jojoba oil/g, particularly preferably from about 0.4 to about 0.6 g jojoba oil/g, is particularly preferred.

For example, in order to enable a uniform suspension of the fibers in the cosmetic composition or in order to increase the cosmetic effect of the composition, the compositions can comprise surfactant as a further optional constituent. The use of non-ionic or cationic surfactants is preferred.

Preferred cationic surfactants are selected from the group of quaternary ammonium compounds. Preferred quaternary ammonium compounds are, in turn, ammonium halides, in particular chlorides and bromides, such as alkyl trimethyl ammonium chlorides, dialkyl dimethyl ammonium chlorides and trialkyl methyl ammonium chlorides, for example cetyl trimethyl ammonium chloride, stearyl trimethyl ammonium chloride, distearyl dimethyl ammonium chloride, lauryl dimethyl ammonium chloride, lauryl dimethyl benzyl ammoniumchloride and tricetyl methyl ammonium chloride, and the imidazolium compounds known under the INCI names Quaternium-27 and Quaternium-83. The use of cetyl trimethyl ammonium chloride is very particularly preferred.

The weight share of the surfactant in the total weight of the cosmetic composition is preferentially from about 0.05 to about 2.0% by weight, preferably from about 0.1 to about 1.5% by weight, and in particular from about 0.2 to about 1.0% by weight.

The composition of some further preferred cosmetic compositions can be deduced from the following overview 2 (values in % by weight relate to the total weight of the cosmetic composition).

| | Formula 1 | Formula 2 | Formula 3 | Formula 4 | Formula 5 |
|---|---|---|---|---|---|
| Hydrophobized fibers | 0.1 to 5.0 | 0.1 to 2.0 | 0.1 to 2.0 | 0.1 to 1.0 | 0.1 to 0.5 |
| Ethanol | 20 to 50 | 20 to 50 | 20 to 45 | 25 to 40 | 25 to 40 |
| Polymer | 0.5 to 8.0 | 0.5 to 8.0 | 1.0 to 6.0 | 1.0 to 6.0 | 2.0 to 4.0 |
| Propellant | 30 to 70 | 35 to 65 | 35 to 65 | 40 to 60 | 40 to 60 |
| Optional additives | to 100 | to 100 | to 100 | to 100 | to 100 |

| | Formula 1a | Formula 2a | Formula 3a | Formula 4a | Formula 5a |
|---|---|---|---|---|---|
| Hydrophobized fibers | 0.1 to 5.0 | 0.1 to 2.0 | 0.1 to 2.0 | 0.1 to 1.0 | 0.1 to 0.5 |
| Ethanol | 20 to 50 | 20 to 50 | 20 to 45 | 25 to 40 | 25 to 40 |
| Polyvinylpyrrolidone, | 0.5 to 8.0 | 0.5 to 8.0 | 1.0 to 6.0 | 1.0 to 6.0 | 2.0 to 4.0 |
| vinylpyrrolidone/vinyl | | | | | |
| acetate copolymer | | | | | |
| Propellant | 30 to 70 | 35 to 65 | 35 to 65 | 40 to 60 | 40 to 60 |
| Optional additives | to 100 | to 100 | to 100 | to 100 | to 100 |

| | Formula 1b | Formula 2b | Formula 3b | Formula 4b | Formula 5b |
|---|---|---|---|---|---|
| Hydrophobized fibers | 0.1 to 5.0 | 0.1 to 2.0 | 0.1 to 2.0 | 0.1 to 1.0 | 0.1 to 0.5 |
| Ethanol | 20 to 50 | 20 to 50 | 20 to 45 | 25 to 40 | 25 to 40 |
| N-tert-octylacrylamide, | 0.5 to 8.0 | 0.5 to 8.0 | 1.0 to 6.0 | 1.0 to 6.0 | 2.0 to 4.0 |
| acrylic acid, tert.- | | | | | |
| butylaminoethyl | | | | | |
| methacrylate copolymer | | | | | |
| Propellant | 30 to 70 | 35 to 65 | 35 to 65 | 40 to 60 | 40 to 60 |
| Optional additives | to 100 | to 100 | to 100 | to 100 | to 100 |

| | Formula 6 | Formula 7 | Formula 8 | Formula 9 | Formula 10 |
|---|---|---|---|---|---|
| Hydrophobized | 0.1 to 5.0 | 0.1 to 2.0 | 0.1 to 2.0 | 0.1 to 1.0 | 0.1 to 0.5 |
| cellulose fibers | | | | | |
| Ethanol | 20 to 50 | 20 to 50 | 20 to 45 | 25 to 40 | 25 to 40 |
| Polymer | 0.5 to 8.0 | 0.5 to 8.0 | 1.0 to 6.0 | 1.0 to 6.0 | 2.0 to 4.0 |
| Propellant | 30 to 70 | 35 to 65 | 35 to 65 | 40 to 60 | 40 to 60 |
| Optional additives | to 100 | to 100 | to 100 | to 100 | to 100 |

| | Formula 6a | Formula 7a | Formula 8a | Formula 9a | Formula 10a |
|---|---|---|---|---|---|
| Hydrophobized | 0.1 to 5.0 | 0.1 to 2.0 | 0.1 to 2.0 | 0.1 to 1.0 | 0.1 to 0.5 |
| cellulose fibers | | | | | |
| Ethanol | 20 to 50 | 20 to 50 | 20 to 45 | 25 to 40 | 25 to 40 |
| Polyvinylpyrrolidone, | 0.5 to 8.0 | 0.5 to 8.0 | 1.0 to 6.0 | 1.0 to 6.0 | 2.0 to 4.0 |
| vinylpyrrolidone/vinyl | | | | | |
| acetate copolymer | | | | | |
| Propellant | 30 to 70 | 35 to 65 | 35 to 65 | 40 to 60 | 40 to 60 |
| Optional additives | to 100 | to 100 | to 100 | to 100 | to 100 |

| | Formula 6b | Formula 7b | Formula 8b | Formula 9b | Formula 10b |
|---|---|---|---|---|---|
| Hydrophobized | 0.1 to 5.0 | 0.1 to 2.0 | 0.1 to 2.0 | 0.1 to 1.0 | 0.1 to 0.5 |
| cellulose fibers | | | | | |
| Ethanol | 20 to 50 | 20 to 50 | 20 to 45 | 25 to 40 | 25 to 40 |
| N-tert-octylacrylamide, | 0.5 to 8.0 | 0.5 to 8.0 | 1.0 to 6.0 | 1.0 to 6.0 | 2.0 to 4.0 |
| acrylic acid, tert.- | | | | | |
| butylaminoethyl | | | | | |
| methacrylate copolymer | | | | | |
| Propellant | 30 to 70 | 35 to 65 | 35 to 65 | 40 to 60 | 40 to 60 |
| Optional additives | to 100 | to 100 | to 100 | to 100 | to 100 |
| | Formula 11 | Formula 12 | Formula 13 | Formula 14 | Formula 15 |
| Hydrophobized fibers | 0.1 to 5.0 | 0.1 to 2.0 | 0.1 to 2.0 | 0.1 to 1.0 | 0.1 to 0.5 |
| with a fiber length | | | | | |
| between 1.0 and 200 μm | | | | | |
| Ethanol | 20 to 50 | 20 to 50 | 20 to 45 | 25 to 40 | 25 to 40 |
| Polymer | 0.5 to 8.0 | 0.5 to 8.0 | 1.0 to 6.0 | 1.0 to 6.0 | 2.0 to 4.0 |
| Propellant | 30 to 70 | 35 to 65 | 35 to 65 | 40 to 60 | 40 to 60 |
| Optional additives | to 100 | to 100 | to 100 | to 100 | to 100 |
| | Formula 11a | Formula 12a | Formula 13a | Formula 14a | Formula 15a |
| Hydrophobized fibers | 0.1 to 5.0 | 0.1 to 2.0 | 0.1 to 2.0 | 0.1 to 1.0 | 0.1 to 0.5 |
| with a fiber length | | | | | |
| between 1.0 and 200 μm | | | | | |
| Ethanol | 20 to 50 | 20 to 50 | 20 to 45 | 25 to 40 | 25 to 40 |
| Polyvinylpyrrolidone, | 0.5 to 8.0 | 0.5 to 8.0 | 1.0 to 6.0 | 1.0 to 6.0 | 2.0 to 4.0 |
| vinylpyrrolidone/vinyl | | | | | |
| acetate copolymer | | | | | |
| Propellant | 30 to 70 | 35 to 65 | 35 to 65 | 40 to 60 | 40 to 60 |
| Optional additives | to 100 | to 100 | to 100 | to 100 | to 100 |
| | Formula 11b | Formula 12b | Formula 13b | Formula 14b | Formula 15b |
| Hydrophobized fibers | 0.1 to 5.0 | 0.1 to 2.0 | 0.1 to 2.0 | 0.1 to 1.0 | 0.1 to 0.5 |
| with a fiber length | | | | | |
| between 1.0 and 200 μm | | | | | |
| Ethanol | 20 to 50 | 20 to 50 | 20 to 45 | 25 to 40 | 25 to 40 |
| N-tert-octylacrylamide, | 0.5 to 8.0 | 0.5 to 8.0 | 1.0 to 6.0 | 1.0 to 6.0 | 2.0 to 4.0 |
| acrylic acid, tert.- | | | | | |
| butylaminoethyl | | | | | |
| methacrylate copolymer | | | | | |
| Propellant | 30 to 70 | 35 to 65 | 35 to 65 | 40 to 60 | 40 to 60 |
| Optional additives | to 100 | to 100 | to 100 | to 100 | to 100 |

| | Formula 16 | Formula 17 | Formula 18 | Formula 19 | Formula 20 |
|---|---|---|---|---|---|
| Hydrophobized | 0.1 to 5.0 | 0.1 to 2.0 | 0.1 to 2.0 | 0.1 to 1.0 | 0.1 to 0.5 |
| cellulose fibers with | | | | | |
| a fiber length between | | | | | |
| 1.0 and 200 μm | | | | | |
| Ethanol | 20 to 50 | 20 to 50 | 20 to 45 | 25 to 40 | 25 to 40 |
| Polymer | 0.5 to 8.0 | 0.5 to 8.0 | 1.0 to 6.0 | 1.0 to 6.0 | 2.0 to 4.0 |
| Propellant | 30 to 70 | 35 to 65 | 35 to 65 | 40 to 60 | 40 to 60 |
| Optional additives | to 100 | to 100 | to 100 | to 100 | to 100 |
| | Formula 16a | Formula 17a | Formula 18a | Formula 19 | Formula 20a |
| Hydrophobized | 0.1 to 5.0 | 0.1 to 2.0 | 0.1 to 2.0 | 0.1 to 1.0 | 0.1 to 0.5 |
| cellulose fibers with | | | | | |
| a fiber length between | | | | | |
| 1.0 and 200 μm | | | | | |
| Ethanol | 20 to 50 | 20 to 50 | 20 to 45 | 25 to 40 | 25 to 40 |
| Polyvinylpyrrolidone, | 0.5 to 8.0 | 0.5 to 8.0 | 1.0 to 6.0 | 1.0 to 6.0 | 2.0 to 4.0 |
| vinylpyrrolidone/vinyl | | | | | |
| acetate copolymer | | | | | |
| Propellant | 30 to 70 | 35 to 65 | 35 to 65 | 40 to 60 | 40 to 60 |
| Optional additives | to 100 | to 100 | to 100 | to 100 | to 100 |

| | Formula 16b | Formula 17b | Formula 18b | Formula 19b | Formula 20b |
|---|---|---|---|---|---|
| Hydrophobized | 0.1 to 5.0 | 0.1 to 2.0 | 0.1 to 2.0 | 0.1 to 1.0 | 0.1 to 0.5 |
| cellulose fibers with | | | | | |
| a fiber length between | | | | | |
| 1.0 and 200 μm | | | | | |
| Ethanol | 20 to 50 | 20 to 50 | 20 to 45 | 25 to 40 | 25 to 40 |
| N-tert-octylacrylamide, | 0.5 to 8.0 | 0.5 to 8.0 | 1.0 to 6.0 | 1.0 to 6.0 | 2.0 to 4.0 |
| acrylic acid, tert.- | | | | | |
| butylaminoethyl | | | | | |
| methacrylate copolymer | | | | | |
| Propellant | 30 to 70 | 35 to 65 | 35 to 65 | 40 to 60 | 40 to 60 |
| Optional additives | to 100 | to 100 | to 100 | to 100 | to 100 |

| | Formula 21 | Formula 22 | Formula 23 | Formula 24 | Formula 25 |
|---|---|---|---|---|---|
| Hydrophobized fibers | 0.1 to 5.0 | 0.1 to 2.0 | 0.1 to 2.0 | 0.1 to 1.0 | 0.1 to 0.5 |
| Ethanol | 20 to 50 | 20 to 50 | 20 to 45 | 25 to 40 | 25 to 40 |
| Polymer | 0.5 to 8.0 | 0.5 to 8.0 | 1.0 to 6.0 | 1.0 to 6.0 | 2.0 to 4.0 |
| Dimethyl ether | 30 to 70 | 35 to 65 | 35 to 65 | 40 to 60 | 40 to 60 |
| Optional additives | to 100 | to 100 | to 100 | to 100 | to 100 |
| | Formula 21a | Formula 22a | Formula 23a | Formula 24a | Formula 25a |
| Hydrophobized fibers | 0.1 to 5.0 | 0.1 to 2.0 | 0.1 to 2.0 | 0.1 to 1.0 | 0.1 to 0.5 |
| Ethanol | 20 to 50 | 20 to 50 | 20 to 45 | 25 to 40 | 25 to 40 |
| Polyvinylpyrrolidone, | 0.5 to 8.0 | 0.5 to 8.0 | 1.0 to 6.0 | 1.0 to 6.0 | 2.0 to 4.0 |
| vinylpyrrolidone/vinyl | | | | | |
| acetate copolymer | | | | | |
| Dimethyl ether | 30 to 70 | 35 to 65 | 35 to 65 | 40 to 60 | 40 to 60 |
| Optional additives | to 100 | to 100 | to 100 | to 100 | to 100 |

| | Formula 21b | Formula 22b | Formula 23b | Formula 24b | Formula 25b |
|---|---|---|---|---|---|
| Hydrophobized fibers | 0.1 to 5.0 | 0.1 to 2.0 | 0.1 to 2.0 | 0.1 to 1.0 | 0.1 to 0.5 |
| Ethanol | 20 to 50 | 20 to 50 | 20 to 45 | 25 to 40 | 25 to 40 |
| N-tert-octylacrylamide, | 0.5 to 8.0 | 0.5 to 8.0 | 1.0 to 6.0 | 1.0 to 6.0 | 2.0 to 4.0 |
| acrylic acid, tert.- | | | | | |
| butylaminoethyl | | | | | |
| methacrylate copolymer | | | | | |
| Dimethyl ether | 30 to 70 | 35 to 65 | 35 to 65 | 40 to 60 | 40 to 60 |
| Optional additives | to 100 | to 100 | to 100 | to 100 | to 100 |

| | Formula 26 | Formula 27 | Formula 28 | Formula 29 | Formula 30 |
|---|---|---|---|---|---|
| Hydrophobized | 0.1 to 5.0 | 0.1 to 2.0 | 0.1 to 2.0 | 0.1 to 1.0 | 0.1 to 0.5 |
| cellulose fibers | | | | | |
| Ethanol | 20 to 50 | 20 to 50 | 20 to 45 | 25 to 40 | 25 to 40 |
| Polymer | 0.5 to 8.0 | 0.5 to 8.0 | 1.0 to 6.0 | 1.0 to 6.0 | 2.0 to 4.0 |
| Dimethyl ether | 30 to 70 | 35 to 65 | 35 to 65 | 40 to 60 | 40 to 60 |
| Optional additives | to 100 | to 100 | to 100 | to 100 | to 100 |

| | Formula 26a | Formula 27a | Formula 28a | Formula 29a | Formula 30a |
|---|---|---|---|---|---|
| Hydrophobized | 0.1 to 5.0 | 0.1 to 2.0 | 0.1 to 2.0 | 0.1 to 1.0 | 0.1 to 0.5 |
| cellulose fibers | | | | | |
| Ethanol | 20 to 50 | 20 to 50 | 20 to 45 | 25 to 40 | 25 to 40 |
| Polyvinylpyrrolidone, | 0.5 to 8.0 | 0.5 to 8.0 | 1.0 to 6.0 | 1.0 to 6.0 | 2.0 to 4.0 |
| vinylpyrrolidone/vinyl | | | | | |
| acetate copolymer | | | | | |
| Dimethyl ether | 30 to 70 | 35 to 65 | 35 to 65 | 40 to 60 | 40 to 60 |
| Optional additives | to 100 | to 100 | to 100 | to 100 | to 100 |

| | Formula 26b | Formula 27b | Formula 28b | Formula 29b | Formula 30b |
|---|---|---|---|---|---|
| Hydrophobized | 0.1 to 5.0 | 0.1 to 2.0 | 0.1 to 2.0 | 0.1 to 1.0 | 0.1 to 0.5 |
| cellulose fibers | | | | | |
| Ethanol | 20 to 50 | 20 to 50 | 20 to 45 | 25 to 40 | 25 to 40 |
| N-tert-octylacrylamide, | 0.5 to 8.0 | 0.5 to 8.0 | 1.0 to 6.0 | 1.0 to 6.0 | 2.0 to 4.0 |
| acrylic acid, tert.- | | | | | |
| butylaminoethyl | | | | | |
| methacrylate copolymer | | | | | |
| Dimethyl ether | 30 to 70 | 35 to 65 | 35 to 65 | 40 to 60 | 40 to 60 |
| Optional additives | to 100 | to 100 | to 100 | to 100 | to 100 |
| | Formula 31 | Formula 32 | Formula 33 | Formula 34 | Formula 35 |
| Hydrophobized fibers | 0.1 to 5.0 | 0.1 to 2.0 | 0.1 to 2.0 | 0.1 to 1.0 | 0.1 to 0.5 |
| with a fiber length | | | | | |
| between 1.0 and 200 μm | | | | | |
| Ethanol | 20 to 50 | 20 to 50 | 20 to 45 | 25 to 40 | 25 to 40 |
| Polymer | 0.5 to 8.0 | 0.5 to 8.0 | 1.0 to 6.0 | 1.0 to 6.0 | 2.0 to 4.0 |
| Dimethyl ether | 30 to 70 | 35 to 65 | 35 to 65 | 40 to 60 | 40 to 60 |
| Optional additives | to 100 | to 100 | to 100 | to 100 | to 100 |
| | Formula 31a | Formula 32a | Formula 33a | Formula 34a | Formula 35a |
| Hydrophobized fibers | 0.1 to 5.0 | 0.1 to 2.0 | 0.1 to 2.0 | 0.1 to 1.0 | 0.1 to 0.5 |
| with a fiber length | | | | | |
| between 1.0 and 200 μm | | | | | |
| Ethanol | 20 to 50 | 20 to 50 | 20 to 45 | 25 to 40 | 25 to 40 |
| Polyvinylpyrrolidone, | 0.5 to 8.0 | 0.5 to 8.0 | 1.0 to 6.0 | 1.0 to 6.0 | 2.0 to 4.0 |
| vinylpyrrolidone/vinyl | | | | | |
| acetate copolymer | | | | | |
| Dimethyl ether | 30 to 70 | 35 to 65 | 35 to 65 | 40 to 60 | 40 to 60 |
| Optional additives | to 100 | to 100 | to 100 | to 100 | to 100 |
| | Formula 31b | Formula 32b | Formula 33b | Formula 34b | Formula 35b |
| Hydrophobized fibers | 0.1 to 5.0 | 0.1 to 2.0 | 0.1 to 2.0 | 0.1 to 1.0 | 0.1 to 0.5 |
| with a fiber length | | | | | |
| between 1.0 and 200 μm | | | | | |
| Ethanol | 20 to 50 | 20 to 50 | 20 to 45 | 25 to 40 | 25 to 40 |
| N-tert-octylacrylamide, | 0.5 to 8.0 | 0.5 to 8.0 | 1.0 to 6.0 | 1.0 to 6.0 | 2.0 to 4.0 |
| acrylic acid, tert.- | | | | | |
| butylaminoethyl | | | | | |
| methacrylate copolymer | | | | | |
| Dimethyl ether | 30 to 70 | 35 to 65 | 35 to 65 | 40 to 60 | 40 to 60 |
| Optional additives | to 100 | to 100 | to 100 | to 100 | to 100 |

| | Formula 36 | Formula 37 | Formula 38 | Formula 39 | Formula 40 |
|---|---|---|---|---|---|
| Hydrophobized | 0.1 to 5.0 | 0.1 to 2.0 | 0.1 to 2.0 | 0.1 to 1.0 | 0.1 to 0.5 |
| cellulose fibers with | | | | | |
| a fiber length between | | | | | |
| 1.0 and 200 μm | | | | | |
| Ethanol | 20 to 50 | 20 to 50 | 20 to 45 | 25 to 40 | 25 to 40 |
| Polymer | 0.5 to 8.0 | 0.5 to 8.0 | 1.0 to 6.0 | 1.0 to 6.0 | 2.0 to 4.0 |
| Dimethyl ether | 30 to 70 | 35 to 65 | 35 to 65 | 40 to 60 | 40 to 60 |
| Optional additives | to 100 | to 100 | to 100 | to 100 | to 100 |
| | Formula 36a | Formula 37a | Formula 38a | Formula 39a | Formula 40a |
| Hydrophobized | 0.1 to 5.0 | 0.1 to 2.0 | 0.1 to 2.0 | 0.1 to 1.0 | 0.1 to 0.5 |
| cellulose fibers with | | | | | |
| a fiber length between | | | | | |
| 1.0 and 200 μm | | | | | |
| Ethanol | 20 to 50 | 20 to 50 | 20 to 45 | 25 to 40 | 25 to 40 |
| Polyvinylpyrrolidone, | 0.5 to 8.0 | 0.5 to 8.0 | 1.0 to 6.0 | 1.0 to 6.0 | 2.0 to 4.0 |
| vinylpyrrolidone/vinyl | | | | | |
| acetate copolymer | | | | | |
| Dimethyl ether | 30 to 70 | 35 to 65 | 35 to 65 | 40 to 60 | 40 to 60 |
| Optional additives | to 100 | to 100 | to 100 | to 100 | to 100 |

| | Formula 36b | Formula 37b | Formula 38b | Formula 39b | Formula 40b |
|---|---|---|---|---|---|
| Hydrophobized | 0.1 to 5.0 | 0.1 to 2.0 | 0.1 to 2.0 | 0.1 to 1.0 | 0.1 to 0.5 |
| cellulose fibers with | | | | | |
| a fiber length between | | | | | |
| 1.0 and 200 μm | | | | | |
| Ethanol | 20 to 50 | 20 to 50 | 20 to 45 | 25 to 40 | 25 to 40 |
| N-tert-octylacrylamide, | 0.5 to 8.0 | 0.5 to 8.0 | 1.0 to 6.0 | 1.0 to 6.0 | 2.0 to 4.0 |
| acrylic acid, tert.- | | | | | |
| butylaminoethyl | | | | | |
| methacrylate copolymer | | | | | |
| Dimethyl ether | 30 to 70 | 35 to 65 | 35 to 65 | 40 to 60 | 40 to 60 |
| Optional additives | to 100 | to 100 | to 100 | to 100 | to 100 |

In an alternative embodiment hydrophilized fibers are used in the cosmetic products. Corresponding fibers have an increased capacity for water absorption compared to the non-hydrophilized fibers.

The water absorption capacity (20° C.) of preferred fibers, particularly of preferred hydrophilized fibers, lies preferably above about 5.0% by weight, preferably above about 7.0% by weight, and in particular above about 9.0% of the net weight of the fibers.

The hydrophilization of the fibers improves their water absorption capacity, amongst other things. Corresponding fibers are therefore used in particular in freshening compositions.

The composition of some further preferred cosmetic compositions can be deduced from the following overview 3 (values in % by weight relate to the total weight of the cosmetic composition).

| | Formula 1 | Formula 2 | Formula 3 | Formula 4 | Formula 5 |
|---|---|---|---|---|---|
| Hydrophilized Fibers | 0.1 to 5.0 | 0.1 to 2.0 | 0.1 to 2.0 | 0.1 to 1.0 | 0.1 to 0.5 |
| Ethanol | 20 to 50 | 20 to 50 | 20 to 45 | 25 to 40 | 25 to 40 |
| Polymer | 0.5 to 8.0 | 0.5 to 8.0 | 1.0 to 6.0 | 1.0 to 6.0 | 2.0 to 4.0 |
| Propellant | 30 to 70 | 35 to 65 | 35 to 65 | 40 to 60 | 40 to 60 |
| Optional additives | to 100 | to 100 | to 100 | to 100 | to 100 |

| | Formula 1a | Formula 2a | Formula 3a | Formula 4a | Formula 5a |
|---|---|---|---|---|---|
| Hydrophilized Fibers | 0.1 to 5.0 | 0.1 to 2.0 | 0.1 to 2.0 | 0.1 to 1.0 | 0.1 to 0.5 |
| Ethanol | 20 to 50 | 20 to 50 | 20 to 45 | 25 to 40 | 25 to 40 |
| Polyvinylpyrrolidone, | 0.5 to 8.0 | 0.5 to 8.0 | 1.0 to 6.0 | 1.0 to 6.0 | 2.0 to 4.0 |
| vinylpyrrolidone/vinyl | | | | | |
| acetate copolymer | | | | | |
| Propellant | 30 to 70 | 35 to 65 | 35 to 65 | 40 to 60 | 40 to 60 |
| Optional additives | to 100 | to 100 | to 100 | to 100 | to 100 |

| | Formula 1b | Formula 2b | Formula 3b | Formula 4b | Formula 5b |
|---|---|---|---|---|---|
| Hydrophilized Fibers | 0.1 to 5.0 | 0.1 to 2.0 | 0.1 to 2.0 | 0.1 to 1.0 | 0.1 to 0.5 |
| Ethanol | 20 to 50 | 20 to 50 | 20 to 45 | 25 to 40 | 25 to 40 |
| N-tert-octylacrylamide, | 0.5 to 8.0 | 0.5 to 8.0 | 1.0 to 6.0 | 1.0 to 6.0 | 2.0 to 4.0 |
| acrylic acid, tert.- | | | | | |
| butylaminoethyl | | | | | |
| methacrylate copolymer | | | | | |
| Propellant | 30 to 70 | 35 to 65 | 35 to 65 | 40 to 60 | 40 to 60 |
| Optional additives | to 100 | to 100 | to 100 | to 100 | to 100 |

| | Formula 6 | Formula 7 | Formula 8 | Formula 9 | Formula 10 |
|---|---|---|---|---|---|
| Hydrophilized | 0.1 to 5.0 | 0.1 to 2.0 | 0.1 to 2.0 | 0.1 to 1.0 | 0.1 to 0.5 |
| cellulose fibers | | | | | |
| Ethanol | 20 to 50 | 20 to 50 | 20 to 45 | 25 to 40 | 25 to 40 |
| Polymer | 0.5 to 8.0 | 0.5 to 8.0 | 1.0 to 6.0 | 1.0 to 6.0 | 2.0 to 4.0 |
| Propellant | 30 to 70 | 35 to 65 | 35 to 65 | 40 to 60 | 40 to 60 |
| Optional additives | to 100 | to 100 | to 100 | to 100 | to 100 |

| | Formula 6a | Formula 7a | Formula 8a | Formula 9a | Formula 10a |
|---|---|---|---|---|---|
| Hydrophilized | 0.1 to 5.0 | 0.1 to 2.0 | 0.1 to 2.0 | 0.1 to 1.0 | 0.1 to 0.5 |
| cellulose fibers | | | | | |
| Ethanol | 20 to 50 | 20 to 50 | 20 to 45 | 25 to 40 | 25 to 40 |
| Polyvinylpyrrolidone, | 0.5 to 8.0 | 0.5 to 8.0 | 1.0 to 6.0 | 1.0 to 6.0 | 2.0 to 4.0 |
| vinylpyrrolidone/vinyl | | | | | |
| acetate copolymer | | | | | |
| Propellant | 30 to 70 | 35 to 65 | 35 to 65 | 40 to 60 | 40 to 60 |
| Optional additives | to 100 | to 100 | to 100 | to 100 | to 100 |

| | Formula 6b | Formula 7b | Formula 8b | Formula 9b | Formula 10b |
|---|---|---|---|---|---|
| Hydrophilized | 0.1 to 5.0 | 0.1 to 2.0 | 0.1 to 2.0 | 0.1 to 1.0 | 0.1 to 0.5 |
| cellulose fibers | | | | | |
| Ethanol | 20 to 50 | 20 to 50 | 20 to 45 | 25 to 40 | 25 to 40 |
| N-tert-octylacrylamide, | 0.5 to 8.0 | 0.5 to 8.0 | 1.0 to 6.0 | 1.0 to 6.0 | 2.0 to 4.0 |
| acrylic acid, tert.- | | | | | |
| butylaminoethyl | | | | | |
| methacrylate copolymer | | | | | |
| Propellant | 30 to 70 | 35 to 65 | 35 to 65 | 40 to 60 | 40 to 60 |
| Optional additives | to 100 | to 100 | to 100 | to 100 | to 100 |
| | Formula 11 | Formula 12 | Formula 13 | Formula 14 | Formula 15 |
| Hydrophilized fibers | 0.1 to 5.0 | 0.1 to 2.0 | 0.1 to 2.0 | 0.1 to 1.0 | 0.1 to 0.5 |
| with a fiber length | | | | | |
| between 1.0 and 200 μm | | | | | |
| Ethanol | 20 to 50 | 20 to 50 | 20 to 45 | 25 to 40 | 25 to 40 |
| Polymer | 0.5 to 8.0 | 0.5 to 8.0 | 1.0 to 6.0 | 1.0 to 6.0 | 2.0 to 4.0 |
| Propellant | 30 to 70 | 35 to 65 | 35 to 65 | 40 to 60 | 40 to 60 |
| Optional additives | to 100 | to 100 | to 100 | to 100 | to 100 |
| | Formula 11a | Formula 12a | Formula 13a | Formula 14a | Formula 15a |
| Hydrophilized fibers | 0.1 to 5.0 | 0.1 to 2.0 | 0.1 to 2.0 | 0.1 to 1.0 | 0.1 to 0.5 |
| with a fiber length | | | | | |
| between 1.0 and 200 μm | | | | | |
| Ethanol | 20 to 50 | 20 to 50 | 20 to 45 | 25 to 40 | 25 to 40 |
| Polyvinylpyrrolidone, | 0.5 to 8.0 | 0.5 to 8.0 | 1.0 to 6.0 | 1.0 to 6.0 | 2.0 to 4.0 |
| vinylpyrrolidone/vinyl | | | | | |
| acetate copolymer | | | | | |
| Propellant | 30 to 70 | 35 to 65 | 35 to 65 | 40 to 60 | 40 to 60 |
| Optional additives | to 100 | to 100 | to 100 | to 100 | to 100 |
| | Formula 11b | Formula 12b | Formula 13b | Formula 14b | Formula 15b |
| Hydrophilized fibers | 0.1 to 5.0 | 0.1 to 2.0 | 0.1 to 2.0 | 0.1 to 1.0 | 0.1 to 0.5 |
| with a fiber length | | | | | |
| between 1.0 and 200 μm | | | | | |
| Ethanol | 20 to 50 | 20 to 50 | 20 to 45 | 25 to 40 | 25 to 40 |
| N-tert-octylacrylamide, | 0.5 to 8.0 | 0.5 to 8.0 | 1.0 to 6.0 | 1.0 to 6.0 | 2.0 to 4.0 |
| acrylic acid, tert.- | | | | | |
| butylaminoethyl | | | | | |
| methacrylate copolymer | | | | | |
| Propellant | 30 to 70 | 35 to 65 | 35 to 65 | 40 to 60 | 40 to 60 |
| Optional additives | to 100 | to 100 | to 100 | to 100 | to 100 |

| | Formula 16 | Formula 17 | Formula 18 | Formula 19 | Formula 20 |
|---|---|---|---|---|---|
| Hydrophilized | 0.1 to 5.0 | 0.1 to 2.0 | 0.1 to 2.0 | 0.1 to 1.0 | 0.1 to 0.5 |
| cellulose fibers with | | | | | |
| a fiber length between | | | | | |
| 1.0 and 200 μm | | | | | |
| Ethanol | 20 to 50 | 20 to 50 | 20 to 45 | 25 to 40 | 25 to 40 |
| Polymer | 0.5 to 8.0 | 0.5 to 8.0 | 1.0 to 6.0 | 1.0 to 6.0 | 2.0 to 4.0 |
| Propellant | 30 to 70 | 35 to 65 | 35 to 65 | 40 to 60 | 40 to 60 |
| Optional additives | to 100 | to 100 | to 100 | to 100 | to 100 |
| | Formula 16a | Formula 17a | Formula 18a | Formula 19 | Formula 20a |
| Hydrophilized | 0.1 to 5.0 | 0.1 to 2.0 | 0.1 to 2.0 | 0.1 to 1.0 | 0.1 to 0.5 |
| cellulose fibers with | | | | | |
| a fiber length between | | | | | |
| 1.0 and 200 μm | | | | | |
| Ethanol | 20 to 50 | 20 to 50 | 20 to 45 | 25 to 40 | 25 to 40 |
| Polyvinylpyrrolidone, | 0.5 to 8.0 | 0.5 to 8.0 | 1.0 to 6.0 | 1.0 to 6.0 | 2.0 to 4.0 |
| vinylpyrrolidone/vinyl | | | | | |
| acetate copolymer | | | | | |
| Propellant | 30 to 70 | 35 to 65 | 35 to 65 | 40 to 60 | 40 to 60 |
| Optional additives | to 100 | to 100 | to 100 | to 100 | to 100 |

| | Formula 16b | Formula 17b | Formula 18b | Formula 19b | Formula 20b |
|---|---|---|---|---|---|
| Hydrophilized | 0.1 to 5.0 | 0.1 to 2.0 | 0.1 to 2.0 | 0.1 to 1.0 | 0.1 to 0.5 |
| cellulose fibers with | | | | | |
| a fiber length between | | | | | |
| 1.0 and 200 μm | | | | | |
| Ethanol | 20 to 50 | 20 to 50 | 20 to 45 | 25 to 40 | 25 to 40 |
| N-tert-octylacrylamide, | 0.5 to 8.0 | 0.5 to 8.0 | 1.0 to 6.0 | 1.0 to 6.0 | 2.0 to 4.0 |
| acrylic acid, tert.- | | | | | |
| butylaminoethyl | | | | | |
| methacrylate copolymer | | | | | |
| Propellant | 30 to 70 | 35 to 65 | 35 to 65 | 40 to 60 | 40 to 60 |
| Optional additives | to 100 | to 100 | to 100 | to 100 | to 100 |

| | Formula 21 | Formula 22 | Formula 23 | Formula 24 | Formula 25 |
|---|---|---|---|---|---|
| Hydrophilized Fibers | 0.1 to 5.0 | 0.1 to 2.0 | 0.1 to 2.0 | 0.1 to 1.0 | 0.1 to 0.5 |
| Ethanol | 20 to 50 | 20 to 50 | 20 to 45 | 25 to 40 | 25 to 40 |
| Polymer | 0.5 to 8.0 | 0.5 to 8.0 | 1.0 to 6.0 | 1.0 to 6.0 | 2.0 to 4.0 |
| Dimethyl ether | 30 to 70 | 35 to 65 | 35 to 65 | 40 to 60 | 40 to 60 |
| Optional additives | to 100 | to 100 | to 100 | to 100 | to 100 |
| | Formula 21a | Formula 22a | Formula 23a | Formula 24a | Formula 25a |
| Hydrophilized Fibers | 0.1 to 5.0 | 0.1 to 2.0 | 0.1 to 2.0 | 0.1 to 1.0 | 0.1 to 0.5 |
| Ethanol | 20 to 50 | 20 to 50 | 20 to 45 | 25 to 40 | 25 to 40 |
| Polyvinylpyrrolidone, | 0.5 to 8.0 | 0.5 to 8.0 | 1.0 to 6.0 | 1.0 to 6.0 | 2.0 to 4.0 |
| vinylpyrrolidone/vinyl | | | | | |
| acetate copolymer | | | | | |
| Dimethyl ether | 30 to 70 | 35 to 65 | 35 to 65 | 40 to 60 | 40 to 60 |
| Optional additives | to 100 | to 100 | to 100 | to 100 | to 100 |

| | Formula 21b | Formula 22b | Formula 23b | Formula 24b | Formula 25b |
|---|---|---|---|---|---|
| Hydrophilized Fibers | 0.1 to 5.0 | 0.1 to 2.0 | 0.1 to 2.0 | 0.1 to 1.0 | 0.1 to 0.5 |
| Ethanol | 20 to 50 | 20 to 50 | 20 to 45 | 25 to 40 | 25 to 40 |
| N-tert-octylacrylamide, | 0.5 to 8.0 | 0.5 to 8.0 | 1.0 to 6.0 | 1.0 to 6.0 | 2.0 to 4.0 |
| acrylic acid, tert.- | | | | | |
| butylaminoethyl | | | | | |
| methacrylate copolymer | | | | | |
| Dimethyl ether | 30 to 70 | 35 to 65 | 35 to 65 | 40 to 60 | 40 to 60 |
| Optional additives | to 100 | to 100 | to 100 | to 100 | to 100 |

| | Formula 26 | Formula 27 | Formula 28 | Formula 29 | Formula 30 |
|---|---|---|---|---|---|
| Hydrophilized | 0.1 to 5.0 | 0.1 to 2.0 | 0.1 to 2.0 | 0.1 to 1.0 | 0.1 to 0.5 |
| cellulose fibers | | | | | |
| Ethanol | 20 to 50 | 20 to 50 | 20 to 45 | 25 to 40 | 25 to 40 |
| Polymer | 0.5 to 8.0 | 0.5 to 8.0 | 1.0 to 6.0 | 1.0 to 6.0 | 2.0 to 4.0 |
| Dimethyl ether | 30 to 70 | 35 to 65 | 35 to 65 | 40 to 60 | 40 to 60 |
| Optional additives | to 100 | to 100 | to 100 | to 100 | to 100 |

| | Formula 26a | Formula 27a | Formula 28a | Formula 29a | Formula 30a |
|---|---|---|---|---|---|
| Hydrophilized | 0.1 to 5.0 | 0.1 to 2.0 | 0.1 to 2.0 | 0.1 to 1.0 | 0.1 to 0.5 |
| cellulose fibers | | | | | |
| Ethanol | 20 to 50 | 20 to 50 | 20 to 45 | 25 to 40 | 25 to 40 |
| Polyvinylpyrrolidone, | 0.5 to 8.0 | 0.5 to 8.0 | 1.0 to 6.0 | 1.0 to 6.0 | 2.0 to 4.0 |
| vinylpyrrolidone/vinyl | | | | | |
| acetate copolymer | | | | | |
| Dimethyl ether | 30 to 70 | 35 to 65 | 35 to 65 | 40 to 60 | 40 to 60 |
| Optional additives | to 100 | to 100 | to 100 | to 100 | to 100 |

| | Formula 26b | Formula 27b | Formula 28b | Formula 29b | Formula 30b |
|---|---|---|---|---|---|
| Hydrophilized | 0.1 to 5.0 | 0.1 to 2.0 | 0.1 to 2.0 | 0.1 to 1.0 | 0.1 to 0.5 |
| cellulose fibers | | | | | |
| Ethanol | 20 to 50 | 20 to 50 | 20 to 45 | 25 to 40 | 25 to 40 |
| N-tert-octylacrylamide, | 0.5 to 8.0 | 0.5 to 8.0 | 1.0 to 6.0 | 1.0 to 6.0 | 2.0 to 4.0 |
| acrylic acid, tert.- | | | | | |
| butylaminoethyl | | | | | |
| methacrylate copolymer | | | | | |
| Dimethyl ether | 30 to 70 | 35 to 65 | 35 to 65 | 40 to 60 | 40 to 60 |
| Optional additives | to 100 | to 100 | to 100 | to 100 | to 100 |
| | Formula 31 | Formula 32 | Formula 33 | Formula 34 | Formula 35 |
| Hydrophilized fibers | 0.1 to 5.0 | 0.1 to 2.0 | 0.1 to 2.0 | 0.1 to 1.0 | 0.1 to 0.5 |
| with a fiber length | | | | | |
| between 1.0 and 200 μm | | | | | |
| Ethanol | 20 to 50 | 20 to 50 | 20 to 45 | 25 to 40 | 25 to 40 |
| Polymer | 0.5 to 8.0 | 0.5 to 8.0 | 1.0 to 6.0 | 1.0 to 6.0 | 2.0 to 4.0 |
| Dimethyl ether | 30 to 70 | 35 to 65 | 35 to 65 | 40 to 60 | 40 to 60 |
| Optional additives | to 100 | to 100 | to 100 | to 100 | to 100 |
| | Formula 31a | Formula 32a | Formula 33a | Formula 34a | Formula 35a |
| Hydrophilized fibers | 0.1 to 5.0 | 0.1 to 2.0 | 0.1 to 2.0 | 0.1 to 1.0 | 0.1 to 0.5 |
| with a fiber length | | | | | |
| between 1.0 and 200 μm | | | | | |
| Ethanol | 20 to 50 | 20 to 50 | 20 to 45 | 25 to 40 | 25 to 40 |
| Polyvinylpyrrolidone, | 0.5 to 8.0 | 0.5 to 8.0 | 1.0 to 6.0 | 1.0 to 6.0 | 2.0 to 4.0 |
| vinylpyrrolidone/vinyl | | | | | |
| acetate copolymer | | | | | |
| Dimethyl ether | 30 to 70 | 35 to 65 | 35 to 65 | 40 to 60 | 40 to 60 |
| Optional additives | to 100 | to 100 | to 100 | to 100 | to 100 |
| | Formula 31b | Formula 32b | Formula 33b | Formula 34b | Formula 35b |
| Hydrophilized fibers | 0.1 to 5.0 | 0.1 to 2.0 | 0.1 to 2.0 | 0.1 to 1.0 | 0.1 to 0.5 |
| with a fiber length | | | | | |
| between 1.0 and 200 μm | | | | | |
| Ethanol | 20 to 50 | 20 to 50 | 20 to 45 | 25 to 40 | 25 to 40 |
| N-tert-octylacrylamide, | 0.5 to 8.0 | 0.5 to 8.0 | 1.0 to 6.0 | 1.0 to 6.0 | 2.0 to 4.0 |
| acrylic acid, tert.- | | | | | |
| butylaminoethyl | | | | | |
| methacrylate copolymer | | | | | |
| Dimethyl ether | 30 to 70 | 35 to 65 | 35 to 65 | 40 to 60 | 40 to 60 |
| Optional additives | to 100 | to 100 | to 100 | to 100 | to 100 |

| | Formula 36 | Formula 37 | Formula 38 | Formula 39 | Formula 40 |
|---|---|---|---|---|---|
| Hydrophilized | 0.1 to 5.0 | 0.1 to 2.0 | 0.1 to 2.0 | 0.1 to 1.0 | 0.1 to 0.5 |
| cellulose fibers with | | | | | |
| a fiber length between | | | | | |
| 1.0 and 200 μm | | | | | |
| Ethanol | 20 to 50 | 20 to 50 | 20 to 45 | 25 to 40 | 25 to 40 |
| Polymer | 0.5 to 8.0 | 0.5 to 8.0 | 1.0 to 6.0 | 1.0 to 6.0 | 2.0 to 4.0 |
| Dimethyl ether | 30 to 70 | 35 to 65 | 35 to 65 | 40 to 60 | 40 to 60 |
| Optional additives | to 100 | to 100 | to 100 | to 100 | to 100 |
| | Formula 36a | Formula 37a | Formula 38a | Formula 39a | Formula 40a |
| Hydrophilized | 0.1 to 5.0 | 0.1 to 2.0 | 0.1 to 2.0 | 0.1 to 1.0 | 0.1 to 0.5 |
| cellulose fibers with | | | | | |
| a fiber length between | | | | | |
| 1.0 and 200 μm | | | | | |
| Ethanol | 20 to 50 | 20 to 50 | 20 to 45 | 25 to 40 | 25 to 40 |
| Polyvinylpyrrolidone, | 0.5 to 8.0 | 0.5 to 8.0 | 1.0 to 6.0 | 1.0 to 6.0 | 2.0 to 4.0 |
| vinylpyrrolidone/vinyl | | | | | |
| acetate copolymer | | | | | |
| Dimethyl ether | 30 to 70 | 35 to 65 | 35 to 65 | 40 to 60 | 40 to 60 |
| Optional additives | to 100 | to 100 | to 100 | to 100 | to 100 |

| | Formula 36b | Formula 37b | Formula 38b | Formula 39b | Formula 40b |
|---|---|---|---|---|---|
| Hydrophilized | 0.1 to 5.0 | 0.1 to 2.0 | 0.1 to 2.0 | 0.1 to 1.0 | 0.1 to 0.5 |
| cellulose fibers with | | | | | |
| a fiber length between | | | | | |
| 1.0 and 200 μm | | | | | |
| Ethanol | 20 to 50 | 20 to 50 | 20 to 45 | 25 to 40 | 25 to 40 |
| N-tert-octylacrylamide, | 0.5 to 8.0 | 0.5 to 8.0 | 1.0 to 6.0 | 1.0 to 6.0 | 2.0 to 4.0 |
| acrylic acid, tert.- | | | | | |
| butylaminoethyl | | | | | |
| methacrylate copolymer | | | | | |
| Dimethyl ether | 30 to 70 | 35 to 65 | 35 to 65 | 40 to 60 | 40 to 60 |
| Optional additives | to 100 | to 100 | to 100 | to 100 | to 100 |

In a further alternative embodiment preferred cosmetic compositions contains at least one fragrance.

In particular, additional nourishing substances can be cited as optional further suitable active substances or auxiliaries which can be contained in the cosmetic compositions.

The composition for example can contain at least one protein hydrolyzate and/or one of the derivatives thereof as nourishing substance. Protein hydrolyzates are product mixtures that are obtained by acid-, base- or enzyme-catalyzed degradation of proteins. The term “protein hydrolyzates” is understood as contemplated herein to also mean total hydrolyzates and individual amino acids and derivatives thereof, and mixtures of different amino acids. The molecular weight of the protein hydrolyzates that can be used as contemplated herein lies between about 75, the molecular weight for glycine, and about 200,000, and the molecular weight is preferably from about 75 to about 50,000, and very particularly preferably from about 75 to about 20,000 Dalton.

The cosmetic composition can also contain at least one vitamin, a provitamin, a vitamin precursor and/or one of the derivatives thereof as nourishing substance. Here, preferred vitamins, provitamins and vitamin precursors are those usually assigned to the groups A, B, C, E, F and H.

Further nourishing substances are panthenol, caffeine, nicotinamide and sorbitol.

The cosmetic compositions may also contain at least one plant extract, but also mono- or oligosaccharides and/or lipids as nourishing substance.

In particular, aerosol-dispensing containers are suitable for the dispensing and application of the cosmetic compositions. A further subject matter of the application relates to the corresponding cosmetic product, comprising
a) a cosmetic composition as contemplated herein
b) an aerosol-dispensing container.

The term “aerosol-dispensing container” is understood to mean pressurized containers inside which a higher gas pressure prevails than outside the container and from which a gas flow can be discharged via a valve. The aerosol-dispensing container, in other words, is a pressurized container with the aid of which a product (for example a cosmetic composition) can be dispensed via a valve by way of the internal gas pressure of the container.

The cosmetic compositions can be produced in the conventional manner. Generally, all constituents of the cosmetic composition with the exception of the propellant are filled into a suitable pressure-tight container. This is then sealed using a valve. Lastly, the desired amount of propellant is filled by way of conventional techniques.

Vessels made of metal (aluminum, tinplate, tin), protected or non-splintering plastic or glass, which is coated externally with plastic, are potential pressure-tight containers, wherein the pressure tightness and breaking strength, corrosion resistance, ease of filling and also aesthetic considerations, ease of handling, printability, etc. all play a role in the selection of such vessels. Special interior protective coatings ensure the corrosion resistance in relation to the composition packaged inside the pressurized container. The used valves particularly preferably have an internally-coated valve disc, wherein coating and valve material are compatible with one another. If aluminum valves are used, their valve discs can thus be coated on the inside, for example with a Micoflex coating. If tinplate valves are used, their valve discs can thus be coated on the inside, for example with PET (polyethylene terephthalate).

A multi-chamber dispenser can also be used as aerosol-dispensing container. The multi-chamber dispenser can also be designed such that one chamber is filled with the compressed propellant and another chamber is filled with the rest of the constituents of the composition as contemplated herein. A multi-chamber dispenser of this kind is for example what is known as a bag-in-can packaging.

The spray rate of the cosmetic product is preferably from about 6.5 to about 10.0 g/10 s.

As mentioned further above, the compositions as contemplated herein are suitable in particular for cleaning, freshening and deodorizing keratin fibers. Corresponding uses of the cosmetic compositions are a further subject of this application.

A last subject of this application is a method for the temporary shaping of keratin fibers, in particular human hair, in which method a cosmetic composition as contemplated herein is applied to keratin fibers. That stated above also applies, mutatis mutandis, for the constituents of this composition, weight shares thereof and preferred embodiments thereof.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. A cosmetic composition for cosmetic treatment of keratin fibers, the composition comprising:
(a) fibers,
(b) ethanol,
(c) at least one polymer, and
(d) propellant.

2. The cosmetic composition according to claim 1, further comprising:
(e) from about 0 to about 10% by weight of dye(s), and
(f) from about 0 to about 10% by weight of pigment(s),
wherein a sum of the amounts of ingredients (e) and (f) is from about 0.0001 to about 10% by weight, in relation to the total weight of the cosmetic composition.

3. The cosmetic composition according to claim 1, wherein the fibers are present in an amount, in relation to the total weight of the cosmetic composition, of from about 0.1 to about 5.0% by weight.

4. The cosmetic composition according to claim 1, wherein the fibers are selected from the group of plant fibers.

5. The cosmetic composition according to claim 1, wherein the fibers have a length of from about 1.0 to about 200 μm.

6. The cosmetic composition according claim 1, wherein ethanol is present in an amount, in relation to the total weight of the cosmetic composition, of from about 20 to about 50% by weight.

7. The cosmetic composition according to claim 1, wherein ethanol is present in an amount, in relation to the total weight of the cosmetic composition, of from about 20 to about 45% by weight.

8. The cosmetic composition according to claim 1, wherein the propellant is present in an amount, in relation to the total weight of the cosmetic composition, of from about 30 to about 70% by weight.

9. The cosmetic composition according to claim 2, wherein the dye(s) is present in an amount, in relation to the total weight of the cosmetic composition, of from about 0.0001 to about 10% by weight.

10. The cosmetic composition according to claim 2, wherein the pigment(s) is present in an amount, in relation to the total weight of the cosmetic composition, of from about 0.0001 to about 10% by weight.

11. The cosmetic composition according to claim 2, wherein the pigment(s) includes at least one pigment from the group of CI12490, CI14700, CI14720, CI15510, CI15985, CI45380, CI47005, CI60730, CI61565, CI73360, CI74160, CI77007, CI77019, CI77288, CI77289, CI77491, and CI77492, wherein the total amount of the at least one pigment from the group is present in an amount, in relation to the total weight of the cosmetic composition, of from about 0.0001 to about 10% by weight.

12. The cosmetic composition according to claim 1, further comprising a starch compound present in an amount, in relation to the total weight of the cosmetic composition, of from about 1.0 to about 10% by weight.

13. The cosmetic composition according to claim 12, wherein the ingredients (e) and/or (f) and the starch compound are present in the form of a starch-dye compound and/or a starch-pigment compound.

14. The cosmetic composition according to claim 2, wherein the ingredients (a) and (e) and/or (a) and (f) are present in the form of a fiber-dye compound and/or a fiber-pigment compound.

15. A cosmetic product comprising:
a) a cosmetic composition according to claim 1, and
b) an aerosol-dispensing container.
